(19) United States
(12) Patent Application Publication
(10) Pub. No.: US 2019/0381071 A1
(43) Pub. Date: Dec. 19, 2019

(54) METHODS AND COMPOSITIONS FOR TREATING AND/OR PREVENTING THE PROGRESSION AND/OR ONSET OF AGE-RELATED NEURODEGENERATION

(71) Applicant: Enterin, Inc., Philadelphia,, PA (US)

(72) Inventors: Michael Zasloff, Philadelphia, PA (US); Denise Barbut, Philadelphia, PA (US)

(73) Assignee: Enterin, Inc., Philadelphia,, PA (US)

(21) Appl. No.: 16/440,414

(22) Filed: Jun. 13, 2019

Related U.S. Application Data
(60) Provisional application No. 62/684,496, filed on Jun. 13, 2018.

(51) Int. Cl. A61K 31/575; A61K 9/00; A61P 25/28

(52) U.S. Cl. CPC A61K 31/575; A61P 25/28; A61K 9/0043; A61K 9/0053

(57) ABSTRACT

This invention relates to methods of treating and/or preventing the progression and/or onset of age-related neurodegeneration. The invention also relates to methods of reversibly slowing the growth and/or aging of a subject, and/or extending the potential lifespan of the subject, comprising administration of the naturally occurring aminosterol MSI-1436, or derivatives or salts thereof. Also described are methods of treating, preventing or delaying the onset of age-related diseases or conditions comprising administration of Aminosterol 1436, or derivatives or salts thereof.

### CROSS-REFERENCE TO RELATED APPLICATIONS

This application claims priority to U.S. Provisional Application No. 62/684,496, filed Jun. 13, 2018. The content of this application is incorporated herein by reference in its entirety.

### FIELD OF THE INVENTION

This disclosure relates to new uses of Aminosterol 1436, shown below, and salts and derivatives thereof.

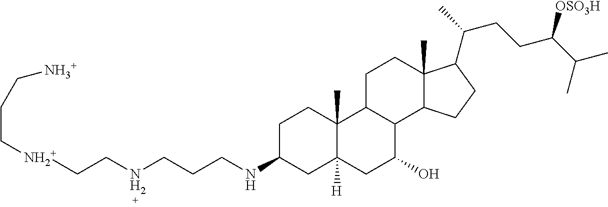

### BACKGROUND OF THE INVENTION

Aminosterol 1436 is an aminosterol isolated from the dogfish shark, which is structurally related to squalamine (U.S. Pat. No. 5,840,936; Rao, Shinnar et al. 2000). It is also known as MSI-1436, trodusquemine and produlestan.

Aminosterol 1436 exhibits antiviral activity against HIV in tissue culture (U.S. Pat. No. 5,763,430) via a mechanism proposed to involve inhibition of a lymphocyte-specific NHE by 1436, resulting in suppression of cytokine responsiveness, and subsequent depression of the capacity of the lymphocyte to support HIV replication (U.S. Pat. No. 5,763,430). Aminosterol 1436, however, has an additional pharmacological property, not shared with squalamine, namely potent appetite suppression and promotion of dose-dependent weight loss (U.S. Pat. No. 6,143,738; Ahima et al. (2002)). In addition, Aminosterol 1436 has been shown to inhibit the phosphatase PTP1B (Lantz et al., 2010) (Ahima et al., 2002).

Several clinical trials have been conducted relating to the use of Aminosterol 1436:

(1) ClinicalTrials.gov Identifier NCT00509132 for “A Phase I, Double-Blind, Randomized, Placebo-Controlled Ascending IV Single-Dose Tolerance and Pharmacokinetic Study of Trodusquemine in Healthy Volunteers,” by Genaera Corp.;

(2) ClinicalTrials.gov Identifier NCT00606112 for “A Single Dose, Tolerance and Pharmacokinetic Study in Obese or Overweight Type 2 Diabetic Volunteer,” by Genaera Corp.;

(3) ClinicalTrials.gov Identifier NCT00806338 for “An Ascending Multi-Dose, Tolerance and Pharmacokinetic Study in Obese or Overweight Type 2 Diabetic Volunteers,” by Genaera Corp.; and

(4) ClinicalTrials.gov Identifier: NCT02524951 for “Safety and Tolerability of MSI-1436C in Metastatic Breast Cancer,” by DepyMed Inc.

### SUMMARY OF THE INVENTION

This disclosure relates to methods of treating and/or preventing neurodegeneration to a subject in need. The method comprises comprising administering a pharmaceutical composition comprising a therapeutically effective amount of Aminosterol 1436 or a pharmaceutically acceptable salt or derivative thereof to the subject. The subject can be an animal or human.

In one embodiment, the neurodegeneration is age-related. In another embodiment, the neurodegeneration is correlated with one or more conditions or diseases selected from the group consisting of age-related dementia, Alzheimer's disease, Parkinson's disease, Lewy Body dementia, fronto temperal dementia, vascular dementia, amyotrophic lateral sclerosis (ALS), multiple sclerosis (MS), multiple system atrophy (MSA), progressive supranuclear palsy (PSP)), olivo-ponto-cerebellar degeneration, or age related cognitive decline without a specific diagnosis from the group above.

In another embodiment, progression or onset of the neurodegeneration is slowed, halted, or reversed over a defined time period following administration of the pharmaceutical composition, as measured by a medically-recognized technique; and/or the neurodegeneration is positively impacted by administration of the pharmaceutical composition; and/or the neurodegeneration is positively impacted by administration of the pharmaceutical composition and the positive impact and/or progression of neurodegeneration is measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; and/or progression or onset of the neurodegeneration is slowed, halted, or reversed over a defined time period following administration of the pharmaceutical composition, as measured by a medically-recognized technique, and the progression or onset of neurodegeneration is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or the neurodegeneration is correlated with abnormal α-synuclein (αS) pathology and/or dopaminergic dysfunction.

In another embodiment, the neurodegeneration is correlated with (a) neural cell death caused by septic shock, intracerebral bleeding, subarachnoidal hemorrhage, multiinfarct dementia, inflammatory diseases, neurotrauma, peripheral neuropathies, polyneuropathies, metabolic encephalopathies, and infections of the central nervous system; or (b) a neurodegenerative disease selected from the group consisting of synucleopathies, Alzheimer's disease, Parkinson's disease, dementia with Lewy bodies, multiple system atrophy, Huntington's disease, multiple sclerosis, parkinsonism, amyotrophic lateral sclerosis (ALS), schizophrenia, Friedreich's ataxia, vascular dementia, spinal muscular atrophy, frontotemporal dementia, supranuclear palsy, progressive supranuclear palsy, progressive nuclear palsy, degenerative processes associated with aging, dementia of aging, Guadeloupian parkinsonism, spinocerebellar ataxia, hallucinations, stroke, traumatic brain injury, down syndrome, Gaucher's disease, Krabbe's disease (KD), lysosomal conditions affecting glycosphingolipid metabolism, cerebral palsy, and epilepsy; or (c) a psychological or behavioral disorder; or (d) a psychological or behavioral disorder which is selected from the group consisting of aberrant motor and obsessive-compulsive behaviors, sleep disorders, REM sleep behavior disorder (RBD), depression, major depressive disorder, agitation, anxiety, delirium, irritability, ADHD, apathy, bipolar disorder, disinhibition, addiction, illusion and delusions, amnesia, and autism; or (e) a cerebral ischemic disorder or a general ischemic disorder; or (f) a cerebral ischemic disorder which is selected from the group consisting of cerebral microangiopathy, intrapartal cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, and diabetic retinopathy; or (g) a general ischemic disorder which is selected from the group consisting of high blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, and pulmonary edema.

The methods of the invention can result in slowing, halting, or reversing progression or onset of the neurodegeneration over a defined time period following administration of the pharmaceutical composition, as measured by a medically-recognized technique. In addition, the neurodegeneration can be positively impacted by administration of the pharmaceutical composition. The positive impact and/or progression of neurodegeneration can be measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis (include clinical examination, sense of smell examination, cognitive testing, sleep studies, circadian rhythm analysis. In addition, the progression or onset of neurodegeneration can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique over a defined period of time.

In another aspect of the invention, the neurodegeneration is correlated with abnormal α-synuclein (αS) pathology and/or dopaminergic dysfunction.

The invention also encompasses methods of treating and/or preventing neurodegeneration wherein the neurodegeneration is correlated with (a) neural cell death caused by septic shock, intracerebral bleeding, subarachnoidal hemorrhage, multiinfarct dementia, inflammatory diseases, neurotrauma, peripheral neuropathies, polyneuropathies, metabolic encephalopathies, and infections of the central nervous system; or (b) a neurodegenerative disease selected from the group consisting of synucleopathies, Alzheimer's disease, Parkinson's disease, dementia with Lewy bodies, multiple system atrophy, Huntington's disease, multiple sclerosis, parkinsonism, amyotrophic lateral sclerosis (ALS), schizophrenia, Friedreich's ataxia, vascular dementia, spinal muscular atrophy, frontotemporal dementia, supranuclear palsy, progressive supranuclear palsy, progressive nuclear palsy, degenerative processes associated with aging, dementia of aging, Guadeloupian parkinsonism, spinocerebellar ataxia, hallucinations, stroke, traumatic brain injury, down syndrome, Gaucher's disease, Krabbe's disease (KD), lysosomal conditions affecting glycosphingolipid metabolism, cerebral palsy, and epilepsy; or (c) a psychological or behavioral disorder; or (d) a psychological or behavioral disorder which is selected from the group consisting of aberrant motor and obsessive-compulsive behaviors, sleep disorders, REM sleep behavior disorder (RBD), depression, major depressive disorder, agitation, anxiety, delirium, irritability, ADHD, apathy, bipolar disorder, disinhibition, addiction, illusion and delusions, amnesia, and autism; or (e) a cerebral ischemic disorder or a general ischemic disorder; or (f) a cerebral ischemic disorder which is selected from the group consisting of cerebral microangiopathy, intrapartal cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, and diabetic retinopathy; or (g) a general ischemic disorder which is selected from the group consisting of high blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, and pulmonary edema.

In another embodiment, encompassed are methods of treating, preventing, or delaying the onset of age-related diseases, conditions, or health problems. The method comprises administering to a subject, which can be an animal or human, a pharmaceutical composition comprising a therapeutically effective amount of Aminosterol 1436 or a derivative or salt thereof for a desirable period of time. The age-related disease, condition, or health problem can be, for example, selected from the group consisting of atherosclerosis and cardiovascular disease, cancer, arthritis, cataracts, osteoporosis, diabetes, hypertension, Alzheimer's disease, arthritis, or osteoporosis.

This invention also relates to methods of reversibly slowing the growth and/or aging of a subject, and/or delaying maturation of a subject, and/or extending the potential lifespan of a subject. The subject can be an animal or human. The method comprises administering a pharmaceutical composition comprising a therapeutically effective amount of Aminosterol 1436 or a pharmaceutically acceptable salt or derivative thereof to the subject, wherein the subject has not yet reached maturity.

In one embodiment, the present invention is directed to methods of reversibly slowing the growth and/or maturation of a subject, which can be an animal or human, comprising administering a pharmaceutical composition comprising a therapeutically effective amount of Aminosterol 1436 or a pharmaceutically acceptable salt or derivative thereof to the animal, wherein the subject has not yet reached maturity.

In another embodiment, encompassed is a method of reversibly slowing or delaying the growth, maturation, and/or aging of a subject, and/or extending the potential lifespan of the subject, comprising administering to the subject a pharmaceutical composition comprising a therapeutically effective amount of Aminosterol 1436 or a pharmaceutically acceptable salt or derivative thereof. The slowed or delayed growth can be measured by height and/or weight, as compared to a subject the same age and sex, who is not administered the pharmaceutical composition comprising a therapeutically effective amount of Aminosterol 1436 or a pharmaceutically acceptable salt or derivative thereof. In addition, the subject administered a pharmaceutical composition according to the invention can have delayed or slowed growth, as measured by height and/or weight, as compared to a subject the same age and sex and who is not treated with a method of the invention, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. Further, the delayed maturation can be measured by showing a delay in skeletal maturation. Finally, the subject administered the pharmaceutical composition can have delayed maturation, as measured by skeletal maturation, as compared to an untreated subject which is the same age and sex, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In an exemplary embodiment where the method reversibly slows the growth and/or delays maturation of a subject, the method results in delayed maturation and/or or slowed growth over a defined period of time, as measured by height and/or weight, or as measured by skeletal maturation, as compared to a subject who is not administered a pharmaceutical composition according to the invention, which is about the same sex and age. The delay can be, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The period of time over which growth and/or maturation is measured can be for example, one or more months or one or more years, e.g., about 6 months, about 1 year, about 18 months, about 2 years, about 36 months, about 3, about 4, about 5, about 6, about 7, about 8, about 9, about 10, about 11, about 12, about 13, about 14, about 15, about 16, about 17, about 18, about 19, or about 20 years, or any amount of months or years in between the values of about 6 months to about 20 years or more.

In one embodiment, the present invention is directed methods of retarding the aging process of a subject, which can be an animal or human. The methods of the invention comprise administering a pharmaceutical composition comprising a therapeutically effective amount of Aminosterol 1436 or a pharmaceutically acceptable salt or derivative thereof to the subject, where the subject has either reached maturity or has not yet reached maturity. The characteristics of aging impacted by administration of Aminosterol 1436 or a derivative or salt thereof can be, for example, muscle endurance, coordination, social behavior and cognitive ability.

For example, administration of a pharmaceutical composition according to the invention may result in improving impaired muscle endurance, as compared to an untreated subject, which is the same sex and age, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In another example, administration of a pharmaceutical composition according to the invention may improve impaired coordination, as compared to an untreated subject, which is the same sex and age, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

Finally, administration of a pharmaceutical composition according to the invention may improve impaired cognitive ability, as compared to an untreated subject, which is the same sex and age, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In another embodiment, the present invention is directed to methods of extending the potential lifespan of a subject, which can be an animal or human. In one aspect, the method comprises safely and reversibly retarding the growth rate of the animal or human during a period of development prior to maturity. The methods of the invention delay growth while preserving overall health. The methods of the invention comprise administering a pharmaceutical composition comprising a therapeutically effective amount of Aminosterol 1436 or a pharmaceutically acceptable salt or derivative thereof to the subject, wherein the subject has not yet reached maturity.

In yet another embodiment, the invention is directed to methods of extending the potential lifespan of a subject, which can be an animal or human, comprising administering a pharmaceutical composition comprising a therapeutically effective amount of Aminosterol 1436 or a derivative or salt thereof to the subject, wherein the subject has reached maturity.

In one embodiment, any of the methods of the invention can be administered during a critical “developmental window” of the subject. Preferably, the “developmental window” is prior to the onset of maturity of the subject.

In some embodiments, Aminosterol 1436 is modified through medical chemistry to improve bio-distribution, ease of administration, metabolic stability, or any combination thereof to produce an Aminosterol 1436 derivative or salt useful in the methods of the invention. The aminosterol 1436 or a salt or derivative thereof is a pharmaceutically acceptable grade of the aminosterol 1436 or a salt or derivative thereof.

The pharmaceutical composition can comprise one or more pharmaceutically acceptable carriers or excipients, and can be administered via any pharmaceutically acceptable method. For example, the pharmaceutical composition can be administered intravenously, intradermally, subcutaneously, orally, rectally, sublingually, intrathecally, intranasally, or by inhalation. In an exemplary embodiment, the compositions of the invention are administered intranasally. The pharmaceutical formulation, including a formulation designed for intranasal delivery, can be formulated in a power, liquid formulation, etc. For example, intranasal formulations are designed to deliver drug to the upper nasal cavity, e.g., using a device such as ONZETRA®. The formulation can be a liquid spray, aerosol, powder, etc. In another example, the formulation can be administered as a liquid hydrochloride salt, a solid phosphate salt, or a solid base. It can be delivered into the nasal cavity while allowing inhalation into the lungs or confined to the nasal cavity by occluding communication between nose and nasopharynx.

In addition, the pharmaceutical composition can be formulated into any suitable dosage form, such as liquid dispersions, gels, aerosols, lyophilized formulations, tablets, or capsules. Further, the pharmaceutical composition can be formulated into a controlled release formulations, fast melt formulations, delayed release formulations, extended release formulations, pulsatile release formulations, and mixed immediate release and controlled release formulations.

Further, the pharmaceutical composition can be administered via any pharmaceutically acceptable method; and/or the pharmaceutical composition can be administered intravenously, intradermally, subcutaneously, orally, rectally, sublingually, intrathecally, intranasally, or by inhalation; and/or the pharmaceutical composition can be administered intranasally; and/or the pharmaceutical composition can be formulated for oral administration in a composition which is a liquid, capsule, or tablet designed to disintegrate in either the stomach, upper small intestine, or more distal portions of the intestine; and/or the pharmaceutical composition can be formulated for intranasal administration in a composition which is a dry powder nasal spray or liquid nasal spray; and/or the pharmaceutical composition can be formulated into a dosage form selected from the group consisting of liquid dispersions, gels, aerosols, lyophilized formulations, tablets, and capsules; and/or the pharmaceutical composition can be formulated into a dosage form selected from the group consisting of controlled release formulations, fast melt formulations, delayed release formulations, extended release formulations, pulsatile release formulations, and mixed immediate release and controlled release formulations.

Any therapeutically effective dosage of Aminosterol 1436 or a salt or derivative thereof can be used in the methods of the invention. For example, the dosage of Aminosterol 1436 or a derivative or salt thereof can be selected from the group consisting of about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10, about 11, about 12, about 13, about 14, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, or about 150 mg/kg (e.g., dose based upon the weight of the subject to be treated).

The dosage of Aminosterol 1436 or a derivative or salt thereof can be selected from the group consisting of about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10, about 11, about 12, about 13, about 14, about 15, about 20, about 25, about 30, about 35, about 40, about 45, about and 50 mg/kg; and/or about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, or about 110 mg/m²; and/or selected from the group consisting of about 10 mg to about 400 mg, or about 50 mg to about 350 mg, or about 100 mg to about 300 mg, or about 100 mg to about 200 mg.

In another example, the dosage of Aminosterol 1436 or a derivative or salt thereof can be selected from the group consisting of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, or about 110 mg/m² (e.g., dose based upon the body surface area of the subject to be treated).

In another embodiment, the dosage of Aminosterol 1436 or a derivative or salt thereof can be selected from the group consisting of about 10 mg to about 400 mg, or about 50 mg to about 350 mg, or about 100 mg to about 300 mg, or about 100 mg to about 200 mg, or any amount in-between these values, such as any amount between 10 and 400 mg, any amount between 50 and 350 mg, any amount between 100 and 300 mg, or any amount between 100 and 200 mg.

In yet another embodiment, the disclosure encompasses a method further comprising first determining a dose of the aminosterol 1436 or a salt or derivative thereof for the subject, wherein the aminosterol 1436 dose is determined based on the effectiveness of the aminosterol 1436 dose in improving or resolving a symptom being evaluated, wherein the symptom is related to neurodegeneration, age-related diseases, and/or growth, maturation, and/or aging of the subject; and second followed by administering the dose of the aminosterol 1436 or a salt or derivative thereof to the subject for a defined period of time. The method comprises identifying a symptom to be evaluated, identifying a starting dose of the aminosterol 1436 or a salt or derivative thereof for the subject; and administering an escalating dose of the aminosterol 1436 or a salt or derivative thereof to the subject over a defined period of time until an effective dose is identified, wherein the effective dose is the dose where improvement of the symptom is observed, and fixing the aminosterol 1436 dose at that level in that particular subject. The dose of the aminosterol 1436 or a salt or derivative thereof can reverse dysfunction caused by the neurodegeneration and treats, prevents, improves, and/or resolves the symptom being evaluated; and/or the improvement or resolution of the symptom can be measured using a clinically recognized scale or tool; and/or the improvement or resolution of the symptom can be measured using a clinically recognized scale or tool and the clinical scale or tool is selected from the group consisting of Uniformed Parkinson's Disease Scale (UPDRS), Mini Mental State Examination (MMSE), Mini Mental Parkinson (MMP), Informant Questionnaire on Cognitive Decline in the Elderly (IQCODE), The 7-Minute Screen, Abbreviated Mental Test Score (AMTS), Cambridge Cognitive Examination (CAMCOG), Clock Drawing Test (CDT), General Practitioner Assessment of Cognition (GPCOG), Mini-Cog, Memory Impairment Screen (MIS), Montreal Cognitive Assessment (MoCA), Rowland Universal Dementia Assessment (RUDA), Self-Administered Gerocognitive Examination (SAGE), Short and Sweet Screening Instrument (SAS-SI), Short Blessed Test (SBT), St. Louis Mental Status (SLUMS), Short Portable Mental Status Questionnaire (SPMSQ), Short Test of Mental Status (STMS), Time and Change Test (T&C), Test Your Memory (TYM) test, and Addenbrooke's Cognitive Examination-Revised (ACER); and/or the improvement in the symptom can be at least about 3%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale or tool.

In yet another embodiment, the aminosterol 1436 or a salt or derivative thereof is administered orally and the starting dose ranges from about 1 mg up to about 175 mg/day; the starting oral dose is about 25 mg/day; the dose of the for the subject following dose escalation is fixed at a range of from about 1 mg up to about 500 mg/day; the dose of the following dose escalation is fixed at a dose of about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 mg/day; the starting oral aminosterol 1436 dose is about 10, about 15, about 20, about 25, about 30, about 35, about 40, about 45, about 60, about 65, about 70, or about 75 mg/day; and/or the dose of the aminosterol 1436 or a salt or derivative thereof is escalated in about 25 mg increments.

In yet another embodiment, the aminosterol 1436 or a salt or derivative thereof is administered intranasally and the starting dose ranges from about 0.001 mg to about 3 mg/day; the dose for the subject following escalation is fixed at a range of from about 0.001 mg up to about 6 mg/day; the dose following escalation is a dose which is subtherapeutic when administered orally or by injection; and/or the dose is escalated in increments of about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

In yet another embodiment, the dose of the aminosterol 1436 or a salt or derivative thereof is escalated every about 3 to about 5 days; and/or is escalated every about 1 to about 14 days; and/or is escalated every about 1, about 2, about 3, about 4, about 5, about 6, about 7, about 8, about 9, about 10, about 11, about 12, about 13, or about 14 days; and/or is escalated about 1×/week, about 2×/week, about every other week, or about 1×/month; and/or the dose, including the starting or fixed dose, of the aminosterol 1436 or a salt or derivative thereof is administered once per day, every other day, once per week, twice per week, three times per week, four times per week, five times per week, six times per week, every other week, or every few days; and/or the dose, including the starting or fixed dose, of the aminosterol 1436 or a salt or derivative thereof is administered for a first defined period of time of administration, followed by a cessation of administration for a second defined period of time, followed by resuming administration upon recurrence of neurodegeneration or a symptom of neurodegeneration; and/or the dose, including the starting or fixed dose, of the aminosterol 1436 or a salt or derivative thereof is incrementally reduced after the fixed dose of aminosterol or a salt or derivative thereof has been administered to the subject for a defined period of time; and/or the dose, including the starting or fixed dose, of the aminosterol 1436 or a salt or derivative thereof is varied plus or minus a defined amount to enable a modest reduction or increase in the fixed dose; and/or the dose, including the starting or fixed dose, of the aminosterol 1436 or a salt or derivative thereof is varied plus or minus a defined amount to enable a modest reduction or increase in the fixed dose, and the fixed aminosterol 1436 dose is increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%; and/or the starting dose of the aminosterol 1436 or a salt or derivative thereof is higher if the symptom being evaluated is severe, as measured using a clinically recognized scale or tool; and/or the starting aminosterol 1436 dose is based on a baseline score of a cognitive test or tool, wherein if the baseline score correlates with an assessment of mild neurodegeneration, then the starting aminosterol 1436 dose is lower than if the baseline score correlates with an assessment of severe neurodegeneration; and/or the subject experiences moderate or mild neurodegeneration as determined by a clinical scale or test, and wherein the starting oral aminosterol 1436 dose is from about 10 to about 75 mg/day; and/or the subject experiences severe neurodegeneration as determined by a clinical scale or test, and wherein the starting oral aminosterol 1436 dose is greater than about 75 mg/day.

In yet another embodiment, the symptom is selected from the group consisting of (a) cognitive impairment as determined by an IQ score; (b) cognitive impairment as determined by a memory or cognitive function test; (c) decline in thinking and reasoning skills; (d) confusion; (e) poor motor coordination; (f) loss of short term memory; (g) loss of long term memory; (h) identity confusion; (i) impaired judgement; (j) forgetfulness; (k) depression; (l) anxiety; (m) irritability; (n) obsessive-compulsive behavior; (o) apathy and/or lack of motivation; (p) emotional imbalance; (q) problem solving ability; (r) impaired language; (s) impaired reasoning; (t) impaired decision-making ability; (u) impaired ability to concentrate; (v) impaired communication; (w) impaired ability to conduct routine tasks such as cooking; (x) self-care, including feeding and dressing; (y) constipation; (z) neurodegeneration; (aa) sleep problem, sleep disorder, and/or sleep disturbance; (bb) hypertension; (cc) hypotension; (dd) sexual dysfunction; (ee) cardiovascular disease; (ff) cardiovascular dysfunction; (gg) difficulty with working memory; (hh) gastrointestinal (GI) disorders; (ii) attention deficit and hyperactivity disorder; (jj) seizures; (kk) urinary dysfunction; (ll) difficulty with mastication; (mm) vision problems; and (nn) muscle weakness.

In yet another embodiment, the symptom to be evaluated is (a) cognitive impairment as determined by an IQ score or as determined by a memory or cognitive function test and wherein: (i) progression or onset of the CI is slowed, halted, or reversed over a defined period of time following administration of the fixed escalated dose of the aminosterol 1436 or a salt or derivative thereof, as measured by a medically-recognized technique; (ii) the CI is positively impacted by the fixed escalated dose of the aminosterol 1436 or a salt or derivative thereof, as measured by a medically-recognized technique; (iii) the CI is positively impacted by the fixed escalated dose of the aminosterol 1436 or a salt or derivative thereof, as measured by a medically-recognized technique and the positive impact on and/or progression of cognitive decline is measured quantitatively or qualitatively by one or more medically-recognized techniques selected from the group consisting of ADASCog, Mini-Mental State Exam (MMSE), Mini-cog test, Woodcock-Johnson Tests of Cognitive Abilities, Leiter International Performance Scale, Miller Analogies Test, Raven's Progressive Matrices, Wonderlic Personnel Test, IQ tests, or a computerized tested selected from Cantab Mobile, Cognigram, Cognivue, Cognision, and Automated Neuropsychological Assessment Metrics Cognitive Performance Test (CPT); and/or (iv) the progression or onset of CI is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; or (b) depression, wherein: (i) the method results in improvement in a subject's depression, as measured by one or more clinically-recognized depression rating scale; (ii) the method results in improvement in a subject's depression, as measured by one or more clinically-recognized depression rating scale and the improvement is in one or more depression characteristics selected from the group consisting of mood, behavior, bodily functions such as eating, sleeping, energy, and sexual activity, and/or episodes of sadness or apathy; and/or (iii) the method results in improvement in a subject's depression, as measured by one or more clinically-recognized depression rating scale, and the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%, and optionally wherein the one or more clinically-recognized depression rating scale is selected from the group consisting of the Patient Health Questionnaire-9 (PHQ-9); the Beck Depression Inventory (BDI); Zung Self-Rating Depression Scale; Center for Epidemiologic Studies-Depression Scale (CES-D); and the Hamilton Rating Scale for Depression (HRSD); (c) constipation, wherein: (i) treating the constipation prevents and/or delays the onset and/or progression of the neurodegeneration; (ii) the fixed escalated aminosterol 1436 dose causes the subject to have a bowel movement; (iii) the method results in an increase in the frequency of bowel movement in the subject; (iv) the method results in an increase in the frequency of bowel movement in the subject and the increase in the frequency of bowel movement is defined as: (1) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (2) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; (iv) as a result of the method the subject has the frequency of bowel movement recommended by a medical authority for the age group of the subject; and/or (v) the starting aminosterol 1436 dose is determined by the severity of the constipation, wherein: (1) if the average complete spontaneous bowel movement (CSBM) or spontaneous bowel movement (SBM) is one or less per week, then the starting aminosterol 1436 dose is at least about 150 mg; and (2) if the average CSBM or SBM is greater than one per week, then the starting aminosterol 1436 dose is about 75 mg or less; (d) a sleep problem, sleep disorder, or sleep disturbance and: (i) the sleep problem, sleep disorder, or sleep disturbance comprises a delay in sleep onset, sleep fragmentation, REM-behavior disorder, sleep-disordered breathing including snoring and apnea, day-time sleepiness, micro-sleep episodes, narcolepsy, circadian rhythm dysfunction, REM disturbed sleep, or any combination thereof; (ii) the sleep problem, sleep disorder, or sleep disturbance comprises REM-behavior disorder, which comprises vivid dreams, nightmares, and acting out the dreams by speaking or screaming, or fidgeting or thrashing of arms or legs during sleep; (iii) treating the sleep problem, sleep disorder, or sleep disturbance prevents or delays the onset and/or progression of the neurodegeneration; (iv) the method results in a positive change in the sleeping pattern of the subject; wherein the positive change is defined as: (1) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (2) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (v) as a result of the method the subject obtains the total number of hours of sleep recommended by a medical authority for the age group of the subject; (e) the method of any one of subsections (a)-(d), wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

The subject to be treated with a method according to the invention can be for example, a common pet, such as a dog or cat. In addition, the subject to be treated can be livestock, such as a horse, cattle, goat, sheep, pig or any farm animal. Finally, the subject to be treated can be a human.

Both the foregoing summary and the following description of the drawings and detailed description are exemplary and explanatory. They are intended to provide further details of the invention, but are not to be construed as limiting. Other objects, advantages, and novel features will be readily apparent to those skilled in the art from the following detailed description of the invention.

### DESCRIPTION OF THE FIGURES

FIG. 1: Shows the accumulation of Aminosterol 1436 within the centers of the brain that control growth, maturation, and senescence following intravenous administration to a rat via a peripheral vein, or injected directly into the 3^{rd} ventricle of the brain.

FIG. 2: Shows weight (g) (y axis) vs age (days) (x axis) for three groups of mice administered 10 mg/kg or 5 mg/kg Aminosterol 1436 (MSI-1436), and a control group. While all animals reached the mature weight of about 40 grams, the control animals reached maturity at 120 days, and the animals that received 5 and 10 mg/kg of Aminosterol 1436 reached maturity at 150 and 255 days, respectively.

FIG. 3: Shows a graph of weight (g) (y axis) vs time for animals given either vehicle or 10 mg/kg (i.p.) of Aminosterol 1436 every 3 days for two doses, for a total of 20 mg/kg over a 6 day period. Animals were then weighed and body length measured once weekly for a period of 40 days. At Day 0 animals in the control group had a starting weight (g) of 16 g, while animals in the Aminosterol 1436 group had a weight of 12 g. At day 40, the control group had a weight of 24 g, or an increase of 50%. In contrast, at Day 40 the Aminosterol 1436 group had a weight of 11 g, or a decrease of 8.3%.

### DETAILED DESCRIPTION OF THE INVENTION

### I. Overview of the Invention

This invention relates to methods of treating and/or preventing neurodegeneration to a subject in need. The neurodegeneration may be age-related, and/or may be correlated with a condition such as age-related dementia, Alzheimer's disease, Parkinson's disease, Lewy Body dementia, fronto temperal dementia, vascular dementia, amyotrophic lateral sclerosis (ALS), multiple sclerosis (MS), multiple system atrophy (MSA), progressive supranuclear palsy (PSP)), olivo-ponto-cerebellar degeneration, or age related cognitive decline without a specific diagnosis from the group above. The method comprises administering a pharmaceutical composition comprising a therapeutically effective amount of Aminosterol 1436 or a pharmaceutically acceptable salt or derivative thereof to the subject. The subject can be an animal or human.

The present invention is also directed to methods of delaying maturation, retarding the aging process, and/or increasing the potential lifespan of subject, which can be an animal or human. The invention is also directed to methods of preventing, treating, and/or delaying onset of age-related diseases or conditions in a subject. The methods comprise administering to the subject a pharmaceutical composition comprising a therapeutically effective amount of Aminosterol 1436 or a derivative or salt thereof.

The present invention is based on the discovery of the unexpected and unprecedented activity of Aminosterol 1436 in treating and/or preventing neurodegeneration, including but not limited to age-related neurodegeneration, as well as reversibly retarding the growth rate of subjects, and in particular the early growth rate of subjects. Moreover, the methods of the invention do not negatively impact normal health and behavior of the subjects treated.

Aminosterol 1436 is a naturally occurring aminosterol. Aminosterol 1436 is also known as aminosterol MSI-1436 and trodusquemine. It is believed that Aminosterol 1436 is found in the liver of all sharks. Interestingly, it was recently reported that the longest-living vertebrate known to science is the Greenland shark, which has an estimated lifespan ranging between 252 and 512 years, with 390 the likeliest average. It is hypothesized that the synthesis and presence of Aminosterol 1436 in shark species positively impacts the lifespan of the shark. The Greenland shark grows to maturity very slowly, with growth at about 1 cm/year with a full grown length of about 7 meters.

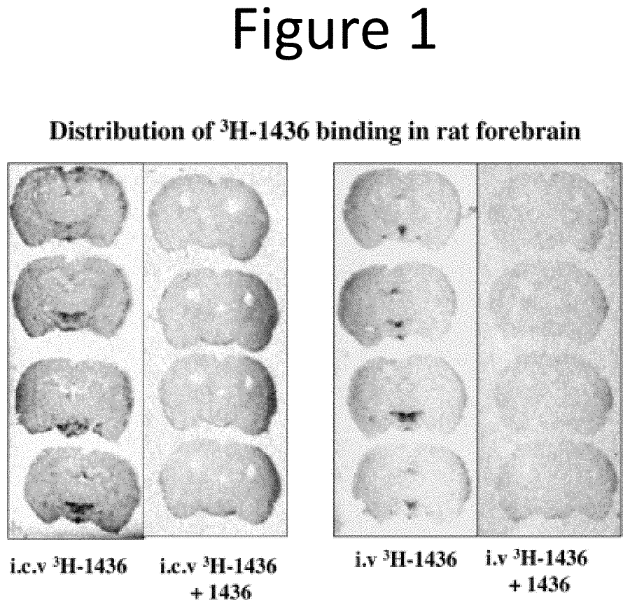

The exact mechanism by which Aminosterol 1436 achieves its effect on preventing, delaying onset, and/or treating neurodegeneration, delaying maturity, retarding aging, and increasing potential lifespan is not known. However, without being bound by theory, Applicant theorizes that the effect of Aminosterol 1436 is likely in part due to the drug's effects on the hypothalamus within the brain of the animal. As seen in FIG. 1, when radioactive Aminosterol 1436 is administered to a rat intravenously via a peripheral vein (IV), or injected directly into the 3^{rd} ventricle of the brain (ICV), the compound accumulates within the centers of the brain that control growth, maturation and senescence.

The hypothalamus is a portion of the brain that contains a number of small nuclei with a variety of functions. One of the most important functions of the hypothalamus is to link the nervous system to the endocrine system via the pituitary gland. The hypothalamus is responsible for the regulation of certain metabolic processes and other activities of the autonomic nervous system.

We have found that treating an adult mouse with Aminosterol 1436 stimulates neuroregeneration, as the number of cells was found to increase in areas known to be associated with regeneration, such as the subependymal zone and granular layers of the olfactory bulb, but also areas not associated with significant neuroregeneration, such as the medial and lateral hypothalamus and thalamus. The hypothalamus is of particular interest because it happens to be the site of localization of Aminosterol 1436 in the brain. Thus, this data shows that Aminosterol 1436 triggers neuroregeneration.

The disclosed methods can be used to treat a range of subjects, including human and non-human animals, including mammals, as well as immature and mature animals, including human children and adults. Examples of livestock that can be treated with the methods of the invention include but are not limited to goats, sheep, horses, rabbits, cattle, chickens and other poultry, pigs, camel, alpaca, llama, etc. Examples of zoo animals that can be treated with the methods of the invention include but are not limited to elephants, lions, tigers, giraffes, etc. Examples of pets that can be treated with methods of the invention include but are not limited to dogs, cats, pigs, ferrets, rabbits, rodents (e,g., gerbils, hamsters, chinchillas, rats, and guinea pigs), and avian pets (e.g., parrots, passerines, and fowl).

Methods of slowing maturation and aging, and extending potential lifespan, are useful for example, in animal husbandry, to extend the potential lifespan of, for example, livestock animals such as horses, cattle, sheep, pigs and goats. The methods of the invention can also be used to slow the growth of common pets such as dogs or cats, maintaining them in a smaller, younger state for a longer period of time than would normally occur. In addition, methods of delaying aging could result in extending the potential lifespan of an animal such as a pet.

The methods of the invention can be administered to animals or humans either prior to maturity or after maturity. The methods of the invention administered to a subject prior to maturity can result in slowed growth, slowed maturation, as well as other results described herein. The methods of the invention administered to a subject after maturity can result in (1) delayed aging; (2) treating, preventing, or delaying onset of age-related diseases and/or conditions; and (3) extending potential lifespan, as well as other results described herein.

In another embodiment, the invention could be used to slow the maturation and aging process in subjects, and additionally extend potential lifespan. In one embodiment, a subject can be treated with a pharmaceutical composition comprising a therapeutically effective amount of Aminosterol 1436 or a derivative or salt thereof prior to maturity and would thereby grow more slowly than an untreated subject, potentially resulting in an extended lifespan, barring unforeseen consequences such as infection, accidents, or organic disease.

The invention could also be used to slow the aging process. The subject would be treated at maturity and beyond with a pharmaceutical composition comprising a therapeutically effective amount of Aminosterol 1436 or a derivative or salt thereof, which can result in a slowed aging process for the subject as compared to an untreated subject, resulting in the treated subject remaining more youthful for a longer period of time. Characteristics of aging that may be impacted by the methods of the invention are described herein.

In another embodiment, encompassed are methods of treating, preventing, or delaying the onset of age-related diseases, conditions, or health problems. The method comprises administering to a subject, such as an animal or human, a pharmaceutical composition comprising a therapeutically effective amount of Aminosterol 1436 or a derivative or salt thereof for a desirable period of time. The age-related disease, condition, or health problem can be, for example, selected from the group consisting of atherosclerosis and cardiovascular disease, cancer, arthritis, cataracts, osteoporosis, diabetes, hypertension, Alzheimer's disease, arthritis, and osteoporosis.

Aminosterol 1436

Aminosterol 1436 is the preferred compound, although any derivative or salt thereof that improves the pharmacological characteristics of the Aminosterol 1436 molecule can be used in the methods of the invention. A derivative of Aminosterol 1436 may have one or more chemical modifications which do not modify the activity of Aminosterol 1436. A “derivative” of Aminosterol 1436 in which modifications well known in the art of medicinal chemistry to “mimic” the original spatial and charge characteristics of a portion of the original structure can be introduced to improve the therapeutic characteristics of the aminosterol. In general, such modifications are introduced to influence metabolism, ease of administration, biodistribution, or any combination thereof. Examples of such variants or derivatives include, but are not limited to, (1) substitutions of the sulfate by a sulfonate, phosphate, carboxylate, or other anionic moiety chosen to circumvent metabolic removal of the sulfate moiety and oxidation of the cholesterol side chain; (2) replacement of a hydroxyl group by a non-metabolizable polar substituent, such as a fluorine atom, to prevent its metabolic oxidation or conjugation; and (3) substitution of various ring hydrogen atoms to prevent oxidative or reductive metabolism of the steroid ring system. The pharmaceutical composition can comprise one or more pharmaceutically acceptable carriers or excipients.

In some embodiments, the methods of the invention can employ a formulation of Aminosterol 1436 (Zasloff, Williams et al. 2001) as an insoluble salt of phosphate, polyphosphate, or an organic phosphate ester. Aminosterol 1436 is shown in Formula II below:

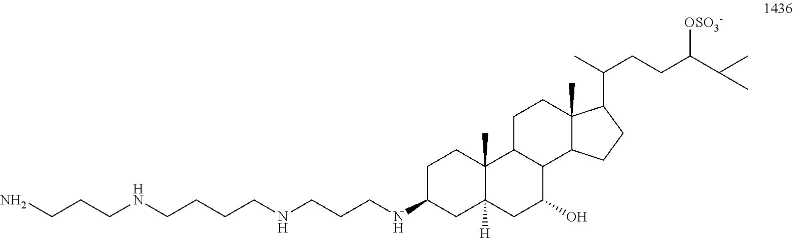

Aminosterol 1436 or its derivatives or salts thereof can be administered via any pharmaceutically acceptable method. For example, the pharmaceutical composition in the methods of the invention can be administered intravenously, intradermally, subcutaneously, orally, rectally, sublingually, intrathecally, intranasally, or by inhalation. Pharmaceutical compositions appropriate for each of the specific routes are utilized.

Developmental Window

In one embodiment, the methods of the invention are administered to a subject, including a human, during a “developmental window” in the life of the subject. For example, administration of the method during a developmental window of the animal can result in reversible slowing of the growth rate and maturation process of the animal. The “developmental window” is from birth to the animal or human reaches maturity as evidenced by ceased growth. In mice, this window extends from weaning to just prior to maturity, e.g., about 4-5 months of age. Comparable windows for other animals would correspond to the periods of growth specific for those animals, as described below.

TABLE 1

| Animal/Human | Developmental Window |
|---|---|
| Human | Humans grow fastest (other than in the womb) as infants and toddlers, |
| | rapidly declining from a maximum at birth to roughly age 2, tapering to |
| | a slowly declining rate, and then during the pubertal growth spurt, a |
| | rapid rise to a second maximum (at around 11-12 years for female, and |
| | 13-14 years for male), followed by a steady decline to zero. On average, |
| | female growth speed trails off to zero at about 15 or 16 years, whereas |
| | the male curve continues for approximately 3 more years, going to zero |
| | at about 18-20. |
| | The developmental window for a male human is from birth to about 18 |
| | years of age. |
| | The developmental window for a female human is from birth to about 15 |
| | years of age. |
| Cat | Cats stop growing when they reach adulthood -- usually around their 1st |
| | birthday. Their bones stop growing at about 8 months old and they begin |
| | sexual maturation, which usually takes another four months. |
| | The developmental window for cats is from birth to about 8 months of |
| | age. |
| Dog | Medium-large dog breeds (Collies, Labrador Retrievers, Boxers) are |
| | fully grown by about 18 months and at their full weight by about 2 years |
| | of age. Giant dog breeds (Great Danes, Mastiffs) may take up to three |
| | years to reach their full weight |
| | The developmental window for dogs is from birth to about 2 years of |
| | age. |
| Cow | The developmental window for cows is from birth to about 2 years of |
| | age. |
| Pig | For food: typically six months from birth to butcher weight (e.g., ~250 lb). |
| | The developmental window for pigs raised for food is from birth to |
| | about six months of age. |
| | Pet: pot bellied pigs don't fully mature until they are about 2 to about3 |
| | years of age |
| | The developmental window for potbellied pigs raised as pets is from |
| | birth to about 2 to about 3 years of age. |

### II. Treatment and/or Prevention of Neurodegeneration

As noted above, this invention relates to methods of treating and/or preventing neurodegeneration to a subject in need, including but not limited to age-related neurodegeneration. The neurodegeneration may also be correlated with age-related dementia, Alzheimer's disease, Parkinson's disease, Lewy Body dementia, fronto temperal dementia, vascular dementia, amyotrophic lateral sclerosis (ALS), multiple sclerosis (MS), multiple system atrophy (MSA), progressive supranuclear palsy (PSP)), olivo-ponto-cerebellar degeneration, or age related cognitive decline without a specific diagnosis from the group above. The method comprises comprising administering a pharmaceutical composition comprising a therapeutically effective amount of Aminosterol 1436 or a pharmaceutically acceptable salt or derivative thereof to the subject. The subject can be an animal or human.

In an exemplary method, Aminosterol 1436 is administered intranasally, although any pharmaceutically acceptable delivery method can be used, as detailed herein.

Age-related neurodegeneration is a significant unsolved problem and challenge. The number of people over 60 years is expected to rise from 841 million in 2013 to more than 2 billion in 2050 (United Nations. World population ageing 2013. United Nations. Department of Economic and Social Affairs Population Division; Available online at: http://www.un.org/en/development/desa/population/publications/pdf/ageing/WorldPopulationAg eingReport2013.pdf). As populations get older, age-related neurodegenerative diseases such as Alzheimer's Disease (AD) and Parkinson's Disease (PD) have become more common. Reitz et al., “Epidemiology of Alzheimer disease,” Nat. Rev. Neurol., 7: 137-152 (2011); Reeve et al., “Ageing and Parkinson's disease: why is advancing age the biggest risk factor?” Ageing Res. Rev., 14: 19-30 (2014)). Even for less common neurodegenerative diseases, such as Amyotrophic Lateral Sclerosis (ALS), this trend seems likely. Beghi et al., “The epidemiology of ALS and the role of population-based registries,” Biochim. Biophys. Acta, 1762: 1150-1157 (2006).

Research to date regarding neurodegenerative diseases has resulted in only modest success. For AD, PD, and ALS, researchers have looked at everything from mis-folded proteins to infectious agents. As a result there are acetyl cholinesterase inhibitors that transiently improve cognition in the early stages of AD (Bond et al., “The effectiveness and cost-effectiveness of donepezil, galantamine, rivastigmine and memantine for the treatment of Alzheimer's disease (review of Technology Appraisal No. 111): a systematic review and economic model,” Health Technol. Assess., 16: 1-470 (2012)), dopamine modifying drugs for the temporary amelioration of motor symptoms in the early stages of PD (Müller, T., “Drug therapy in patients with Parkinson's disease,” Transl. Neurodegener., 1:10 (2012)), and an NMDA antagonist which prolongs life for around 3 months in ALS (Gibson and Bromberg, “Amyotrophic lateral sclerosis: drug therapy from the bench to the bedside,” Semin. Neurol., 32: 173-178 (2012)). However, none of these treatments alters the course of these age-related diseases. They remain incurable.

The World Health Organisation (NIH/WHO (2011). Global Health and Aging. NIH Publication no 11-7737. Available online at: https://www.nia.nih.gov/research/publication/global-health-and-aging/preface) looked at 23 low-to middle-income nations and estimated that their combined loss in economic output between 2006 and 2015 due to age-related diseases was USD84 billion, and the global cost of AD alone in 2010 was estimated at USD604 billion. Wimo et al., “The worldwide economic impact of dementia 2010,” Alzheimers Dement., 9: 1-11 (2013).

Parkinson's Disease is the second most common age-related neurodegenerative disease after Alzheimer's disease. Reeve et al. (2014). Parkinson's disease (PD) affects over 1% of the population over the age of 60, which in the US equates to over 500,000 individuals, while in individuals over the age of 85 this prevalence reaches 5%, highlighting the impact that advancing age has on the risk of developing this condition. Id.

Lewy body dementia (LBD) is a disease associated with abnormal deposits of a protein called alpha-synuclein in the brain. These deposits, called Lewy bodies, affect chemicals in the brain whose changes, in turn, can lead to problems with thinking, movement, behavior, and mood. Frontotemporal dementia (FTD) is a group of related conditions resulting from the progressive degeneration of the temporal and frontal lobes of the brain. These areas of the brain play a significant role in decision-making, behavioral control, emotion and language. Finally, vascular dementia is a decline in thinking skills caused by conditions that block or reduce blood flow to the brain, depriving brain cells of vital oxygen and nutrients.

Multiple sclerosis (MS) is a demyelinating disease in which the insulating covers of nerve cells in the brain and spinal cord are damaged. This damage disrupts the ability of parts of the nervous system to communicate, resulting in a range of signs and symptoms, including physical, mental, and sometimes psychiatric problems. Specific symptoms can include double vision, blindness in one eye, muscle weakness, trouble with sensation, or trouble with coordination. MS takes several forms, with new symptoms either occurring in isolated attacks (relapsing forms) or building up over time (progressive forms). Between attacks, symptoms may disappear completely; however, permanent neurological problems often remain, especially as the disease advances. While the cause is not clear, the underlying mechanism is thought to be either destruction by the immune system or failure of the myelin-producing cells. Proposed causes for this include genetics and environmental factors such as being triggered by a viral infection. MS is usually diagnosed based on the presenting signs and symptoms and the results of supporting medical tests. There is no known cure for multiple sclerosis. Treatments attempt to improve function after an attack and prevent new attacks. Medications used to treat MS, while modestly effective, can have side effects and be poorly tolerated. The long-term outcome is difficult to predict, with good outcomes more often seen in women, those who develop the disease early in life, those with a relapsing course, and those who initially experienced few attacks. Life expectancy is on average 5 to 10 years lower than that of an unaffected population.

Multiple sclerosis is the most common immune-mediated disorder affecting the central nervous system. In 2015, about 2.3 million people were affected globally with rates varying widely in different regions and among different populations. That year about 18,900 people died from MS, up from 12,000 in 1990. The disease usually begins between the ages of 20 and 50 and is twice as common in women as in men.

Multiple sclerosis progression varies from person to person. A variety of tools are useful in assessing whether MS is improving, progressing, or staying about the same. Examples of clinical tools used to assess MS progression include, but are not limited to, Expanded Disability Status Scale (EDSS) (also referred to as the Kurtzke scale), Functional System Score (FSS), Disease Steps (DS), Multiple Sclerosis Functional Composite (MSFC), 9-Hole Peg Test (9-HPT), Ambulation Index (AI), Bladder Control Scale (BLCS), Bowel Control Scale (BWCS), Health Status Questionnaire (SF-36), Impact of Visual Impairment Scale (IVIS), Mental Health Inventory (MHI), Modified Fatigue Impact Scale (MFIS), MOS Modified Social Support Survey (MSSS), MOS Pain Effects Scale (PES), Multiple Sclerosis Quality of Life-54 (MSQOL-54), Multiple Sclerosis Quality of Life Inventory (MSQLI), Paced Auditory Serial Addition Test (PASAT), Perceived Deficits Questionnaire (PDQ), Sexual Satisfaction Scale (SSS), and Timed 25-Foot Walk (T25-FW) (https://www.nationalmssociety.org/For-Professionals/Researchers/Resources-for-Researchers/Clinical-Study-Measures).

Multiple system atrophy (MSA), also known as Shy-Drager syndrome, is a rare neurodegenerative disorder, affecting potentially 15,000 to 50,000 Americans, characterized by tremors, slow movement, muscle rigidity, and postural instability (collectively known as parkinsonism) due to dysfunction of the autonomic nervous system, and ataxia. This is caused by progressive degeneration of neurons in several parts of the brain including the substantia nigra, striatum, inferior olivary nucleus, and cerebellum. MSA includes disorders that historically had been referred to as Shy-Drager syndrome, olivopontocerebellar atrophy, and striatonigral degeneration. Many people affected by MSA experience dysfunction of the autonomic nervous system, which commonly manifests as orthostatic hypotension, impotence, loss of sweating, dry mouth and urinary retention and incontinence. The cause of MSA is uncertain and no specific risk factor has been identified, although research indicates that a prion form of the alpha-synuclein protein may cause the disease. About 55% of MSA cases occur in men, with typical age of onset in the late 50s to early 60s. MSA often presents with some of the same symptoms as Parkinson's disease. However, those with MSA generally show little response to the dopamine medications used to treat Parkinson's disease, and only about 9% of MSA patients with tremor had a true parkinsonian pill-rolling tremor. Currently, there are no treatments to delay the progressive neurodegeneration of MSA, and there is no cure.

A variety of clinical tools can be used to diagnose and measure progression of MSA. Currently used techniques include structural and functional brain imaging (e.g., MRI.\, DaTscan or PET scan), cardiac sympathetic imaging, cardiovascular autonomic testing, olfactory testing, sleep study, urological evaluation, and dysphagia and cognitive assessments.

Progressive supranuclear palsy (PSP; or the Steele-Richardson-Olszewski syndrome) is a degenerative disease involving the gradual deterioration and death of specific volumes of the brain. Males and females are affected approximately equally and there is no racial, geographical or occupational predilection. Approximately six people per 100,000 population have PSP. It has been described as a tauopathy. The initial symptoms in two-thirds of cases are loss of balance, lunging forward when mobilizing, fast walking, bumping into objects or people, and falls. Other common early symptoms are changes in personality, general slowing of movement, and visual symptoms. Later symptoms and signs are dementia (typically including loss of inhibition and ability to organize information), slurring of speech, difficulty swallowing, and difficulty moving the eyes, particularly in the vertical direction. Some of the other signs are poor eyelid function, contracture of the facial muscles, a backward tilt of the head with stiffening of the neck muscles, sleep disruption, urinary incontinence and constipation. The affected brain cells are both neurons and glial cells. The neurons display neurofibrillary tangles, which are clumps of tau protein, a normal part of a brain cell's internal structural skeleton. These tangles are often different from those seen in Alzheimer's disease, but may be structurally similar when they occur in the cerebral cortex. Their chemical composition is usually different, however, and is similar to that of tangles seen in corticobasal degeneration. Tufts of tau protein in astrocytes, or tufted astrocytes, are also considered diagnostic. Unlike globose NFTs, they may be more widespread in the cortex. Lewy bodies are seen in some cases, but it is not clear whether this is a variant or an independent co-existing process, and in some cases PSP can coexist with corticobasal degeneration, Parkinson's and/or Alzheimer's Disease, particularly with older patients. Some consider PSP, corticobasal degeneration, and frontotemporal dementia to be variations of the same disease. Others consider them separate diseases. PSP has been shown occasionally to coexist with Pick's disease. There is no known cure for PSP and management is primarily supportive.

A variety of clinical tools can be used to diagnose and measure progression of PSP. MRI is often done to diagnose PSP. An initial diagnosis is based on the person's medical history and a physical and neurological exam. Diagnostic scans such as magnetic resonance imaging may show shrinkage at the top of the brain stem. Other imaging tests can look at brain activity in known areas of degeneration.

Olivo-ponto-cerebellar degeneration, or Olivopontocerebellar atrophy (OPCA), is the degeneration of neurons in specific areas of the brain—the cerebellum, pons, and inferior olivary nucleus. OPCA is present in several neurodegenerative syndromes, including inherited and non-inherited forms of ataxia (such as the hereditary spinocerebellar ataxia known as Machado-Joseph disease) and multiple system atrophy (MSA), with which it is primarily associated. OPCA may also be found in the brains of individuals with prion disorders and inherited metabolic diseases. The characteristic areas of brain damage that indicate OPCA can be seen by imaging the brain using CT scans or MRI studies. OPCA is characterized by progressive cerebellar ataxia, leading to clumsiness in body movements, veering from midline when walking, wide-based stance, and falls without signs of paralysis or weakness. Clinical presentation can vary greatly between patients, but mostly affects speech, balance and walking. Other possible neurological problems include spasmodic dysphonia, hypertonia, hyperreflexia, rigidity, dysarthria, dysphagia and neck dystonic posture. No specific treatment exists for individuals with OPCA. Treatment is symptomatic and supportive.

A variety of neuroimaging techniques may be useful for the early diagnosis and/or measurement of progression of neurodegenerative disorders. Examples of such techniques include but are not limited to neuroimaging, functional MRI, structural MRI, diffusion tensor imaging (DTI) (including for example diffusion tensor measures of anatomical connectivity), [18F]fluorodeoxyglucose (FDG) PET, agents that label amyloid, [18F]F-dopa PET, radiotracer imaging, volumetric analysis of regional tissue loss, specific imaging markers of abnormal protein deposition (e.g., for AD progression), multimodal imaging, and biomarker analysis. Jon Stoessl, “Neuroimaging in the early diagnosis of neurodegenerative disease,” Transl. Neurodegener., 1: 5 (2012). Combinations of these techniques can also be used to measure disease progression.

For example, structural MRI can be used to measure atrophy of the hippocampus and entorhinal cortex in AD, as well as involvement of the lateral parietal, posterior superior temporal and medial posterior cingulate cortices. In frontotemporal dementias (FTD), structural MRI can show atrophy in frontal or temporal poles. DTI can be used to show abnormal white matter in the parietal lobes of patients with dementia with Lewy bodies (DLB) as compared to AD. Functional MRI may reveal reduced frontal but increased cerebellar activation during performance of a working memory task in FTD compared to AD. In another example, [18F]fluorodeoxyglucose (FDG) PET can show reduced glucose metabolism in parietotemporal cortex in AD. Id.

Progression of neurodegeneration can be measured using well known techniques. For example, an electroencephalogram (EEG) can be used as a biomarker for the presence and progression of a neurodegenerative disease. S. Morairty, “Detecting Neurodegenerative Diseases Before Damage Is Done,” SRI International (Jul. 26, 2013) (https://www.sri.com/blog/detecting-neurodegenerative-diseases). Another exemplary technique that can be used to measure progression of neurodegeneration of MRI. Rocca et al., “The Role of T1-Weighted Derived Measures of Neurodegeneration for Assessing Disability Progression in Multiple Sclerosis,” Front Neurol., 8:433 (Sep. 4, 2017).

ALS, or amyotrophic lateral sclerosis, is a progressive neurodegenerative disease that affects nerve cells in the brain and the spinal cord. Motor neurons reach from the brain to the spinal cord and from the spinal cord to the muscles throughout the body. The progressive degeneration of the motor neurons in ALS eventually leads to their demise. When the motor neurons die, the ability of the brain to initiate and control muscle movement is lost. With voluntary muscle action progressively affected, people may lose the ability to speak, eat, move and breathe. The motor nerves that are affected with ALS are the motor neurons that provide voluntary movements and muscle control. There are two different types of ALS, sporadic and familial. Sporadic, which is the most common form of the disease in the U.S., accounts for 90 to 95 percent of all cases. It may affect anyone, anywhere. Familial ALS (FALS) accounts for 5 to 10 percent of all cases in the U.S. Familial ALS means the disease is inherited. In those families, there is a 50% chance each offspring will inherit the gene mutation and may develop the disease. To date, there has been no cure or treatment that halts or reverses ALS. ALS usually strikes people between the ages of 40 and 70, and it is estimated there are more than 20,000 Americans who have the disease at any given time (although this number fluctuates). For unknown reasons, military veterans are approximately twice as likely to be diagnosed with the disease as the general public.

A variety of clinical measures can be used to assess disease progression in ALS. (S. Rutkove, Neurotherapeutics, 12(2): 384-393 (April 2015)). Examples include but are not limited to (1) clinical measures such as ALSFRS-R (Amyotrophic Lateral Sclerosis Functional Rating Scale), strength testing, FVC (forced vital capacity), and Bulbar-specific measures; (2) electrophysiological measures such as CMAP/NI (compound motor action potential/neurophysiological index), MUNE (motor unit number estimate), EIM (electrical impedance myography), and Excitability testing; (3) muscle imaging, such as MRI (magnetic resonance imaging) and US (ultrasound); and (4) Serum and CSF markers.

In one embodiment of the invention, the progression or onset of a neurodegenerative disorder is slowed or prevented over a defined time period, following administration of a composition according to the invention to a subject in need, as measured by a medically-recognized technique. For example, the progression or onset of a neurodegenerative disorder can be slowed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

The period of time over which the progression or onset of a neurodegenerative disorder is measured can be for example, one or more months or one or more years, e.g., about 6 months, about 1 year, about 18 months, about 2 years, about 36 months, about 3, about 4, about 5, about 6, about 7, about 8, about 9, about 10, about 11, about 12, about 13, about 14, about 15, about 16, about 17, about 18, about 19, or about 20 years, or any amount of months or years in between the values of about 6 months to about 20 years or more.

In another embodiment of the invention, a neurodegenerative disorder may be positively impacted by administering a pharmaceutical composition comprising a therapeutically effective amount of Aminosterol 1436 or a derivative or salt thereof. A “positive impact” includes for example slowing advancement of the condition, improving symptoms, etc.

### III. Treatment of Age-Related Health Conditions/Diseases

In another embodiment of the invention, encompassed are methods of treating, preventing, or delaying the onset of age-related diseases or health conditions. The method comprising administering to a subject, which can be an animal or human, a pharmaceutical composition comprising a therapeutically effective amount of Aminosterol 1436 or a derivative or salt thereof.

Examples of age-related diseases that can be treated, prevented, or onset can be delayed include, but are not limited to, atherosclerosis and cardiovascular disease, cancer, arthritis, cataracts, osteoporosis, type 2 diabetes, and hypertension. The incidence of all of these diseases increases rapidly with aging (increases exponentially with age, in the case of cancer). Of the roughly 150,000 people who die each day across the globe, about two thirds-100,000 per day-die of age-related causes. In industrialized nations, the proportion is higher, reaching 90%.

By age 3 about 30% of rats have had cancer, whereas by age 85 about 30% of humans have had cancer. Humans, dogs and rabbits get Alzheimer's disease, but rodents do not. Elderly rodents typically die of cancer or kidney disease, but not of cardiovascular disease. In humans, the relative incidence of cancer increases exponentially with age for most cancers, but levels off or may even decline by age 60-75 (although colon/rectal cancer continues to increase).

Age related health conditions include but are not limited to arthritis, heart disease, osteoporosis, and diabetes. These age-related health conditions may be positively impacted by administering a pharmaceutical composition comprising a therapeutically effective amount of Aminosterol 1436 or a derivative or salt thereof. A “positive impact” includes for example slowing advancement of the condition, improving symptoms, etc.

Atherosclerosis:

In one embodiment of the invention, the method is directed to treating, preventing, or delaying the onset of atherosclerosis in a subject in need, comprising administering a pharmaceutical composition comprising a therapeutically effective amount of Aminosterol 1436 or a salt or derivative thereof. The subject in need can be at risk of developing atherosclerosis. Disease progression can lead eventually to the occurrence of acute cardiovascular events such as myocardial infarction, unstable angina pectoris and sudden cardiac death.

The level of atherosclerosis can be measured using well known techniques, such as CT scans, which allows visualization of both calcified and noncalcified atherosclerotic plaque in the entire coronary tree (Priester et al., J. of Cardiovascular Computed Tomography, 3(2):S81-S90 (2009)). Other recognized methods of measuring the progression of atherosclerosis include, for example, B-mode ultrasonography, intravascular ultrasonography, computed tomography, and magnetic resonance imaging.

In one embodiment of the invention, the progression or onset of atherosclerosis is slowed or prevented over a defined time period as measured by a medically-recognized technique. For example, the progression or onset of atherosclerosis can be slowed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

Cardiovascular Disease:

Cardiovascular disease (CVD) is a class of diseases that involve the heart or blood vessels. Cardiovascular disease includes coronary artery diseases (CAD) such as angina and myocardial infarction (commonly known as a heart attack). Other CVDs include stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, and venous thrombosis. See “Cardiovascular disease,” Wikipedia (https://en.wikipedia.org/wiki/Cardiovascular_disease).

The level of coronary artery calcification, which is a marker for coronary heart disease, can be measured using well known techniques, such as CT scans, which allows visualization of both calcified and noncalcified atherosclerotic plaque in the entire coronary tree (Priester et al., J. of Cardiovascular Computed Tomography, 3(2):S81-S90 (2009)). Other recognized methods of measuring the progression of coronary artery calcification include, for example, B-mode ultrasonography, intravascular ultrasonography, computed tomography, and magnetic resonance imaging.

In one embodiment of the invention, the progression or onset of coronary heart disease is slowed or prevented over a defined time period as measured by a medically-recognized technique. For example, the progression or onset of atherosclerosis can be slowed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

Arthritis:

Arthritis is a term often used to mean any disorder that affects joints. Symptoms generally include joint pain and stiffness.[2] Other symptoms may include redness, warmth, swelling, and decreased range of motion of the affected joints. Measurement of disease progression depends upon the specific type of arthritis, e.g., osteoarthritis, rheumatoid arthritis, etc. For example, MRIs can be used to assess disease progression for osteoarthritis subjects (to evaluate for example cartilage volume/thickness loss).

In one embodiment of the invention, the progression or onset of arthritis is slowed or prevented over a defined time period as measured by a medically-recognized technique. For example, the progression or onset of arthritis can be slowed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

Cataracts:

A cataract is a clouding of the lens in the eye which leads to a decrease in vision. Cataracts often develop slowly and can affect one or both eyes. Symptoms may include faded colors, blurry vision, halos around light, trouble with bright lights, and trouble seeing at night. This may result in trouble driving, reading, or recognizing faces. Poor vision caused by cataracts may also result in an increased risk of falling and depression. Cataracts cause half of all cases of blindness and 33% of visual impairment worldwide. “Cataract,” Wikipedia (https://en.wikipedia.org/wiki/Cataract). About 20 million people are blind due to cataracts. It is the cause of approximately 5% of blindness in the United States, with more than half the people in the United States having cataracts by the age of 80. Cataracts are most commonly due to aging. Lens proteins denature and degrade over time, and this process is accelerated by diseases such as diabetes mellitus and hypertension.

In one embodiment of the invention, the progression or onset of cataracts are slowed or prevented over a defined time period as measured by a medically-recognized technique. For example, the progression or onset of cataracts can be slowed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

Osteoporosis:

Osteoporosis is a disease where increased bone weakness increases the risk of a broken bone. It is the most common reason for a broken bone among the elderly. Osteoporosis becomes more common with age, and it is more common in women than men. About 15% of white people in their 50s and 70% of those over 80 are affected. It is more common in women than men. Osteoporosis is defined as a bone density of 2.5 standard deviations below that of a young adult. This is typically measured by dual-energy X-ray absorptiometry at the hip. See “Osteoporosis,” Wikipedia (

In one embodiment of the invention, the progression or onset of osteoporosis is slowed or prevented over a defined time period as measured by a medically-recognized technique. For example, the progression or onset of osteoporosis can be slowed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

Hypertension:

Hypertension (HTN or HT), also known as high blood pressure (HBP), is a long-term medical condition in which the blood pressure in the arteries is persistently elevated. Long-term high blood pressure, however, is a major risk factor for coronary artery disease, stroke, heart failure, atrial fibrillation, peripheral vascular disease, vision loss, chronic kidney disease, and dementia. See “Hypertension,” Wikipedia (https://en.wikipedia.org/wiki/Hypertension).

In one embodiment of the invention, the progression or onset of hypertension is slowed or prevented over a defined time period as measured by a medically-recognized technique. For example, the progression or onset of hypertension can be slowed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. A successful result can be, for example, a decrease in blood pressure (e.g., by about 5%, 10%, etc.).

### IV. Measurement of Impact on Growth/Maturation

In one embodiment the invention is directed to methods of delaying growth and/or maturation of a subject, which can be a human or animal, comprising administering a pharmaceutical composition comprising a therapeutically effective amount of Aminosterol 1436 or a derivative or salt thereof to the subject. Administration of the pharmaceutical composition comprising Aminosterol 1436 or a derivative or salt thereof can be limited to a brief period during development of a subject, sufficient to slow the rate of growth. Alternatively, administration of the pharmaceutical composition comprising Aminosterol 1436 can be as a maintenance protocol.

It is well established that otherwise healthy animals that grow slowly during early postnatal development live longer than animals that exhibit a rapid rate of early growth. For example, mice with various pituitary mutations that result in small body size, such as the Ames and Snell dwarf mice, live among the longest of any strain of mouse (Blagosklonny, 2013). Mice that grow slowly during the first 2-3 months of age outlive mice that grow more rapidly during that early period of life (Miller et al., 2002). Suppression of the growth hormone/IGF-1 hormonal axis is believed to be, in part, involved in the delay in early growth (Vanhooren and Libert, 2013) though how a delay in early growth translates into longevity is unknown.

By adjusting the Aminosterol 1436 dosing regimen, the extent of the growth and/or maturation delay can be controlled, with a greater delay occurring with greater doses and longer duration of administration of Aminosterol 1436.

In particular, by the selection of the appropriate dosing regimen of Aminosterol 1436, the growth rate of a subject, which can be an animal or human, can be slowed in a measured fashion from a few % to over 50% as compared to that of an untreated subject, which is the same sex and age. For example, a subject treated at an early age with a dose that reduced growth to 50% normal would take twice as long to reach the size of an untreated subject. In this example, when an untreated sibling reached maturity at 5 months of age, the treated sibling would resemble a 2.5 month old and not reach maturity until 10 months of age. It is theorized that the treated subject with slowed growth will simultaneously age more slowly than the untreated subject and is anticipated to live longer.

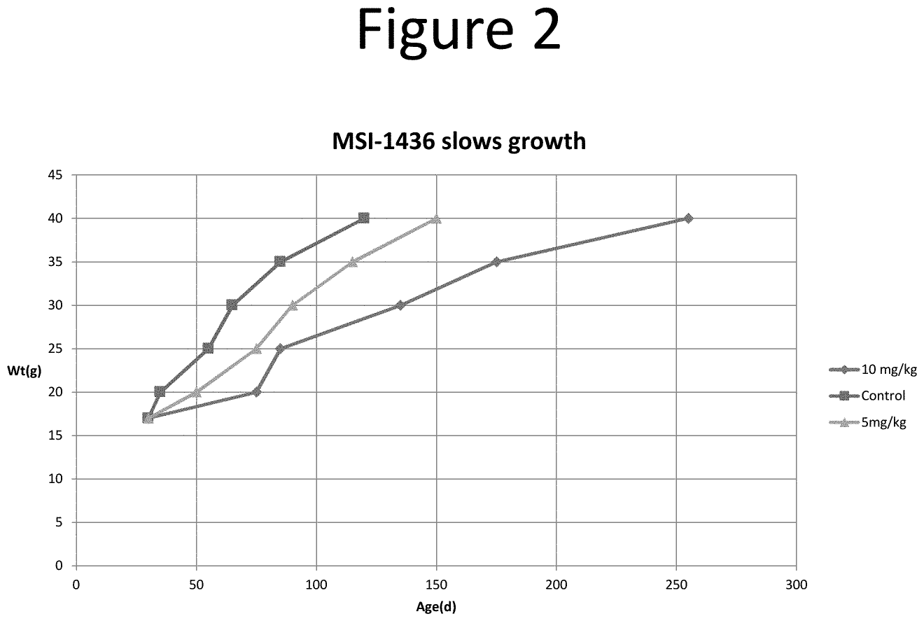

In other embodiments of the invention, administration of Aminosterol 1436 or a derivative or salt thereof delays growth, as measured by height and/or weight, as compared to a subject who is not administered Aminosterol 1436 or a derivative or salt thereof, which is the same sex and age. The delay can be for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The delay in growth can be measured over any time period, and will vary depending upon the subject being treated. See e.g., FIG. 2.

Maturation can be measured in a variety of ways. In humans, maturation is simply the process of children growing and obtaining adult stature. Females tend to mature sooner than boys. Maturation as the process from early childhood, to adolescence and then to full adult stature. Childhood is generally regarded as the time until which one reaches adolescence. The start of adolescence begins with the onset of puberty where hormonal and physical changes begin to occur. Initially, rapid changes begin to occur with increases in height, weight, stature and the development of secondary sex characteristics (Lloyd, R. S., and Oliver, J. L. Strength and Conditioning for Young Athletes: Science and Application. Routledge, 2014. https://www.routledge.com/Strength-and-Conditioning-for-Young-Athletes-Science-and-application/Lloyd-Oliver/p/book/9780415694896). Human biologists usually apply the term “maturity” to level of maturity; that is, the extent to which an individual, or a group of individuals, has proceeded towards adulthood. Therefore, maturation is a particular type of development: development that proceeds to the same end point in all individuals. In this sense, measures relative to adult size for the same individual, for example, present stature as a percentage of actual or predicted adult stature, are measures of maturity. The measurement of skeletal maturity is based on the recognition of maturity indicators; these are visible changes or stages that occur during maturation. Thus, one measurable factor of maturity is carpal and metacarpal bone maturation, which can be readily measured using x-rays. Mohammed et al., The reliability of Fishman method of skeletal maturation for age estimation in children of South Indian population,” J. Nat. Sci. Biol. Med., 5(2):297-302 (2014); and Pichai et al., “A Comparison of Hand Wrist Bone Analysis with Two Different Cervical Vertebral Analysis in Measuring Skeletal Maturation,” J. Int. Oral Health, 6(5):36-41 (2014).

In one embodiment of the invention, administration of Aminosterol 1436 or a derivative or salt thereof delays maturation, as measured by skeletal maturation, as compared to a subject who is not administered Aminosterol 1436 or a derivative or salt thereof, which is the same sex and age. The delay can be for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The delay in maturation can be measured over any time period, and will vary depending upon the subject being treated. For example, a mouse having a typical maturation period of 40 days will have a delay in maturation upon administration of a method according to the invention over a typical period of about 150-300 days, depending upon the dose of Aminosterol 1436 or a derivative or salt thereof administered.

Measurement of Bone Maturation:

Bone age is the degree of maturation of a child's bones. As a person grows from fetal life through childhood, puberty, and finishes growth as a young adult, the bones of the skeleton change in size and shape. These changes can be seen by x-ray. The “bone age” of a child is the average age at which children reach this stage of bone maturation. See “Bone age,” Wikipedia (https://en.wikipedia.org/wiki/Bone_age

At birth, only the metaphyses of the “long bones” are present. The long bones are those that grow primarily by elongation at an epiphysis at one end of the growing bone. The long bones include the femurs, tibias, and fibulas of the lower limb, the humeri, radii, and ulnas of the upper limb (arm+forearm), and the phalanges of the fingers and toes. As a child grows the epiphyses become calcified and appear on the x-rays, as do the carpal and tarsal bones of the hands and feet, separated on the x-rays by a layer of invisible cartilage where most of the growth is occurring. As sex steroid levels rise during puberty, bone maturation accelerates. As growth nears conclusion and attainment of adult height, bones begin to approach the size and shape of adult bones. The remaining cartilaginous portions of the epiphyses become thinner. As these cartilaginous zones become obliterated, the epiphyses are said to be “closed” and no further lengthening of the bones will occur.

### V. Measurement of Impact on Aging

In another embodiment, the invention is directed to methods of retarding the aging process of a subject, which can be an animal or human, comprising administering a pharmaceutical composition comprising a therapeutically effective amount of Aminosterol 1436 or a derivative or salt thereof to the subject. The composition can be administered either prior to or after maturity of the subject.

Characteristics of aging that may be impacted by administration of Aminosterol 1436 or a derivative or salt thereof include, but are not limited to, aspects of aging impacted by the hypothalamus. It has been theorized that the endocrine function of the hypothalamus essentially controls the aging process. T. Hicklin (2017). The hypothalamus is known to regulate important processes including growth, development, reproduction and metabolism. It has also been shown that the hypothalamus regulates aging throughout the body. Hypothalamic stem cells appear to exert their anti-aging effects by releasing molecules called microRNAs (miRNAs). Zhang et al. (2017); Zhang et al. (2013).

In addition, as detailed by Zhang et al. (2017), aspects of aging can be slowed or reversed with administration of miRNA-containing exosomes from hypothalamic stem cells. The present inventors theorize that administration of Aminosterol 1436, or a derivative or salt thereof, has a similar impact.

Delays in aging can be measured by tissue analysis and behavioral testing to assess changes in a subject's age-impaired muscle endurance, coordination, social behavior and cognitive ability. Muscular endurance, which is your ability to use your muscles for extended periods of time at less than their full strength, can be measured using a variety of methods. For example, ACSM (2000) recommends the partial curl-up test to measure endurance of the abdominal muscles and the push-up test to assess endurance of the upper body. Coordination is evaluated by testing the patient's ability to perform rapidly alternating and point-to-point movements correctly. Cognitive ability can be measured using cognitive ability tests. Cognitive ability tests assess abilities involved in thinking (e.g., reasoning, perception, memory, verbal and mathematical ability, and problem solving). Examples of cognitive ability tests include but are not limited to the Cognitive Abilities Test (CogAT), Wechsler Adult Intelligence Scale for adults and the Wechsler Intelligence Scale for Children for school-age test-takers, the Stanford-Binet Intelligence Scales, Woodcock-Johnson Tests of Cognitive Abilities, the Kaufman Assessment Battery for Children, the Cognitive Assessment System, and the Differential Ability Scales.

In one embodiment of the invention, administration of Aminosterol 1436 or a derivative or salt thereof improves impaired muscle endurance, as compared to an untreated subject, which is the same sex and age, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In another embodiment of the invention, administration of Aminosterol 1436 or a derivative or salt thereof improves impaired coordination, as compared to an untreated subject, which is the same sex and age, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In another embodiment of the invention, administration of Aminosterol 1436 or a derivative or salt thereof improves impaired cognitive ability, as compared to an untreated subject, which is the same sex and age, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

### VI. Measurement of Impact on Lifespan

One embodiment of the invention is directed to methods of extending the potential lifespan of a subject, which can be an animal or human. In one aspect the subject has not yet reached maturity, and in another aspect the subject to be treated has reached maturity. The methods comprise administering a pharmaceutical composition comprising a therapeutically effective amount of Aminosterol 1436 or a derivative or salt thereof to the subject.

Lifespan and life expectancy are not synonymous. Life expectancy is defined statistically as the mean number of years remaining for an individual or a group of people at a given age. Id. Life expectancy increases with age as the individual survives the higher mortality rates associated with childhood. Life expectancy is an average for all people in the population—including those who die shortly after birth, those who die in early adulthood (e.g. childbirth, war), and those who live unimpeded until old age. Lifespan is an individual-specific concept—maximum lifespan is therefore an upper bound rather than an average.

In the present invention, an increased lifespan is defined as a lifespan which is greater than life expectancy. For example, a dog administered an Aminosterol 1436 composition according to the invention, and having a life expectancy of about 7 years, is projected to live longer than a dog having the same life expectancy but which is not treated with a method according to the invention. Life expectancies for different animals, breeds of animals, humans in various countries, etc. are all readily available. In an exemplary aspect of the invention, a subject treated with a pharmaceutical composition comprising a therapeutically effective amount of Aminosterol 1436 formulation has an increased lifespan, as compared to a control, of about 5%, about 10%, about 15%, about 20%, about 25%, about 30%, about 35%, about 40%, about 45%, or about 50%. A “control” is defined as an animal which is the same sex, same age, and same type/breed. For a human, a “control” refers to the same sex, same age, same socioeconomic background, and same geographic residence.

### VII. Dosage Forms/Methods of Treatment

Various formulations may be used for administration of the Aminosterol 1436 or derivatives or salts thereof. The formulations may conveniently be presented in unit dosage form and may be prepared by any of the methods well known in the art of pharmacy. Any pharmaceutically acceptable dosage form may be employed in the methods of the invention For example, the composition can be formulated into a dosage form selected from the group consisting of liquid dispersions, gels, aerosols, lyophilized formulations, tablets, capsules, or an intranasal formulations utilizing a powder or liquid. In some embodiments, the Aminosterol 1436 or derivatives or salts thereof may be incorporated into a dosage form selected from the group consisting of controlled release formulations, fast melt formulations, delayed release formulations, extended release formulations, pulsatile release formulations, and mixed immediate release and controlled release formulations. In some embodiments, the dosage form may comprise a combination of the forgoing formulation options (e.g., a controlled release tablet). An exemplary dosage form is a nasal spray. A nasal spray is designed to deliver drug to the upper nasal cavity, and can be a liquid or powder formulation, and in a dosage form such as an aerosol, liquid spray, or powder.

Another exemplary dosage form is an orally administered dosage form, such as a tablet or capsule. These dosage forms can be formulated by any method known in the art. Such methods include the step of bringing into association the Aminosterol 1436 or derivatives or salts thereof with the carrier that constitutes one or more accessory ingredients. In general, the formulations are prepared by uniformly and intimately bringing into association the active ingredient with liquid carriers or finely divided solid carriers or both, and then, if necessary, shaping the product. Another example of an exemplary dosage form is a nasal spray, comprising a dry powder, liquid suspension, liquid emulsion, or other suitable nasal dosage form.

In one embodiment of the invention, an oral dosage form is a liquid, capsule, or tablet designed to disintegrate in either the stomach, upper small intestine, or more distal portions of the intestine with a dissolution rate appropriate to achieve the intended therapeutic benefit.

Formulations or compositions of the invention may be packaged together with, or included in a kit along with instructions or a package insert. Such instructions or package inserts may address recommended storage conditions, such as time, temperature and light, taking into account the shelf-life of the Aminosterol 1436 or derivatives or salts thereof. Such instructions or package inserts may also address the particular advantages of the Aminosterol 1436 or derivatives or salts thereof, such as the ease of storage for formulations that may require use in the field, outside of controlled hospital, clinic or office conditions.

The pharmaceutical composition comprising Aminosterol 1436 or derivatives or salts thereof will be formulated and dosed in a fashion consistent with good medical practice, taking into account the clinical condition of the individual patient, the method of administration, the scheduling of administration, and other factors known to practitioners. The “effective amount” for purposes herein is thus determined by such considerations.

Dosing:

In one embodiment, the dosage of Aminosterol 1436 or a derivative or salt thereof is selected from the group consisting of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 105, 110, 115, 120, 125, 130, 135, 140, 145, or 150 mg/kg. In another embodiment, the dosage of Aminosterol 1436 or a derivative or salt thereof is selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, or 110 mg/m².

In other embodiments of the invention, an effective oral dose generally falls between about 10 mg to about 400 mg, or about 50 mg to about 350 mg, or about 100 mg to about 300 mg, or about 100 mg to about 200 mg. For instance, an effective dose may be about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, or about 400 mg.

Dosing Period:

The pharmaceutical composition comprising Aminosterol 1436 or a derivative or salt thereof can be administered for any suitable period of time, including as a maintenance dose for a prolonged period of time. Dosing can be done on an as needed basis using any pharmaceutically acceptable dosing regimen. For example, dosing can be once or twice daily, once every other day, once every three days, once every four days, once every five days, once every six days, once a week, or divided over multiple time periods during a given day (e.g., twice daily). The dosing schedule may include administration during the morning, midday, or during the evening, or a combination thereof.

In other embodiments, the composition can be administered: (1) as a single dose, or as multiple doses over a period of time; (2) at a maintenance dose for an indefinite period of time; (3) once, twice or multiple times; (4) daily, every other day, every 3 days, weekly, or monthly; (5) for a period of time such as 1, 2, 3, or 4 weeks, 1, 2, 3, 4, 5, 6, 7, 8, 9, 10, 11, or 12 months, 1 year, 1.5 years, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, or 25 years, or (6) any combination of these parameters, such as daily administration for 6 months, weekly administration for 1 or more years, etc.

Exemplary dosing regimens include, but are not limited to: Initiating with a “low” initial daily dose, and gradually increasing the daily dose until a dose is reached that elicits evidence of a measurable impact, e.g., slowed growth rate (e.g., height and weight), improved age-related conditions (e.g., muscle endurance, coordination, social behavior and cognitive ability), or other indicia of desirable effects. In some embodiments, a “low” dose is from about 10 to about 100 mg per person, and the final effective daily dose may be between about 25 to about 1000 mg/person.

Another exemplary dosing regimen includes: Initiating with a “high” initial dose, and reducing the subsequent daily dosing to that required to elicit a desirable response, with the “high” daily dose being between about 50 to about 1000 mg/person, and the subsequent lower daily oral dose being between about 25 to about 500 mg/person.

Yet another exemplary dosing regimen includes periodic dosing, where an effective dose can be delivered once every about 1, about 2, about 3, about 4, about 5, about 6 days, or once weekly, with the initial dose determined to be capable of delaying maturation, retarding the aging process, and/or extending the potential lifespan of a subject, which can be an animal or human.

In some embodiments, the first or initial “large” dose of Aminosterol 1436 or a derivative or salt thereof can be selected from the group consisting of about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1025, about 1050, about 1075, about 1100, about 1125, about 1150, about 1175, about 1200, about 1225, about 1250, about 1275, about 1300, about 1325, about 1350, about 1375, about 1400, about 1425, about 1450, about 1475, about 1500, about 1525, about 1550, about 1575, about 1600, about 1625, about 1650, about 1675, about 1700, about 1725, about 1750, about 1775, about 1800, about 1825, about 1850, about 1875, about 1900, about 1925, about 1950, about 1975, or about 2000 mg. In other embodiments of the invention, the second smaller dose of Aminosterol 1436 or a derivative or salt thereof is less than the first or initial dose and can be selected from the group consisting of about, 10, about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, or about 1000 mg. Finally, in other embodiments of the invention, the periodic Aminosterol 1436 or a derivative or salt thereof dosage (per person) can be selected from the group consisting of about 10, about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, and about 1000 mg.

Any pharmaceutical used for therapeutic administration can be sterile. Sterility is readily accomplished by for example filtration through sterile filtration membranes (e.g., 0.2 micron membranes). Any pharmaceutically acceptable sterility method can be used in the compositions of the invention.

The invention also provides a pharmaceutical pack or kit comprising one or more containers filled with one or more pharmaceutical compositions useful in the disclosed methods of treatment. The kits may include, for instance, containers filled with an appropriate amount of a pharmaceutical composition, either as a powder, to be dissolved, or as a sterile solution, in addition to the Aminosterol 1436 or a derivative or salt thereof. Associated with such container(s) can be a notice in the form prescribed by a governmental agency regulating the manufacture, use or sale of pharmaceuticals or biological products, which notice reflects approval by the agency of manufacture, use or sale for human administration. In addition, the Aminosterol 1436 or a derivative or salt thereof may be employed in conjunction with other therapeutic compounds.

Excipients:

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art. Examples of filling agents include lactose monohydrate, lactose anhydrous, and various starches; examples of binding agents include various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil® 200, talc, stearic acid, magnesium stearate, calcium stearate, and silica gel. Examples of sweeteners may include any natural or artificial sweetener, such as sucrose, xylitol, sodium saccharin, cyclamate, aspartame, and acesulfame. Examples of flavoring agents are Magnasweet® (trademark of MAFCO), bubble gum flavor, and fruit flavors, and the like. Examples of preservatives include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof. Examples of effervescent agents include effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

Optimal oral dosing appears to be on an empty stomach. Aminosterol 1436 or a derivative or salt thereof, for example, is expected to bind tightly to foodstuff, and be unavailable to interact with the intestinal epithelium. Only as the food material is digested is Aminosterol 1436 or a derivative or salt thereof freed.

In another embodiment, the Aminosterol 1436 or a derivative or salt thereof is administered in combination with at least one additional active agent to achieve either an additive or synergistic effect. For example, the additional active agent can be administered via a method selected from the group consisting of (a) concomitantly; (b) as an admixture; (c) separately and simultaneously or concurrently; or (d) separately and sequentially. In another embodiment, the additional active agent is a different aminosterol from that administered in primary method. In yet a further embodiment, the method of the invention comprises administering a first aminosterol which is aminosterol 1436 or a salt or derivative thereof intranasally and administering a second aminosterol which is squalamine or a salt or derivative thereof orally.

For all of the methods of the invention, in one embodiment each dose of Aminosterol 1436 or a derivative or salt thereof is taken on an empty stomach, optionally within about two hours of the subject waking. In another embodiment for all of the methods of the invention, no food is taken or consumed after about 60 to about 90 minutes of taking the dose of Aminosterol 1436 or a derivative or salt thereof. Further, in yet another embodiment applicable to all of the methods of the invention, the Aminosterol 1436 or a derivative or salt thereof can be a pharmaceutically acceptable grade of at least one Aminosterol 1436 or a derivative or salt thereof. For all of the methods of the invention the subject can be a human.

In another embodiment, the subject to be treated according to the methods of the invention can be a member of a patient population at risk for being diagnosed with neurodegeneration.

### VIII. Optional Method of Dose Optimization

Method of Dose Optimization:

In another embodiment, the invention encompasses a method of treating, preventing and/or slowing the onset or neurodegeneration and/or a related symptom in a subject in need. Optionally, the neurodegeneration is correlated with abnormal α-synuclein (aS) pathology and/or dopaminergic dysfunction. The method comprises (a) determining a dose of aminosterol 1436 or a salt or derivative thereof for the subject, wherein the dose of the aminosterol 1436 or a salt or derivative thereof is determined based on the effectiveness of the dose in improving or resolving a neurodegeneration symptom being evaluated, (b) followed by administering the dose to the subject for a period of time, wherein the method comprises (i) identifying a neurodegeneration symptom to be evaluated; (ii) identifying a starting dose of the aminosterol 1436 or a salt or derivative thereof for the subject; and (iii) administering an escalating dose of the aminosterol 1436 or a salt or derivative thereof to the subject over a period of time until an effective dose for the neurodegeneration symptom being evaluated is identified, wherein the effective dose is the aminosterol 1436 dose where improvement or resolution of the neurodegeneration symptom is observed, and fixing the dose at that level for that particular neurodegeneration symptom in that particular subject.

In one embodiment, starting dosages of the aminosterol 1436 or a salt or derivative thereof for oral administration can range, for example, from about 1 mg up to about 175 mg/day, or any amount in-between these two values. An exemplary starting dosage is 25 mg/day. In another embodiment, the composition is administered orally and the dosage is escalated in about 25 mg increments. In yet another embodiment, the composition is administered orally and the dose of aminosterol 1436 or a salt or derivative thereof for the subject following dose escalation is fixed at a range of from about 1 mg up to about 500 mg/day, or any amount in-between these two values. In another aspect, the dose of the aminosterol 1436 or a salt or derivative thereof for the subject following escalation is fixed at a dose of about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 mg/day. In another aspect, the starting oral dose is about 10, about 15, about 20, about 25, about 30, about 35, about 40, about 45, about 60, about 65, about 70, or about 75 mg/day.

In another embodiment, the composition is administered intranasally (IN) and the starting dosage of the aminosterol 1436 or a salt or derivative thereof ranges from about 0.001 mg to about 3 mg/day, or any amount in-between these two values. For example, the starting dosage for IN administration, prior to dose escalation, can be, for example, about 0.001, about 0.005, about 0.01, about 0.02, about 0.03, about 0.05, about 0.06, about 0.07, about 0.08, about 0.09, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 1.0, about 1.1, about 1.25, about 1.3, about 1.4, about 1.5, about 1.6, about 1.7, about 1.75, about 1.8, about 1.9, about 2.0, about 2.1, about 2.25, about 2.3, about 2.4, about 2.5, about 2.6, about 2.7, about 2.75, about 2.8, about 2.9, or about 3 mg/day.

In another embodiment, the composition is administered intranasally and the dosage of the aminosterol 1436 or a salt or derivative thereof is escalated in increments of about 0.01, about 0.05, about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

Finally, in yet another embodiment, the composition is administered intranasally and the dose of the aminosterol 1436 or a salt or derivative thereof for the subject following escalation is fixed at a range of from about 0.001 mg up to about 6 mg/day, or any amount in-between these two values. In yet a further embodiment, the composition is administered intranasally and the dose of the aminosterol 1436 or a salt or derivative thereof for the subject following dose escalation is a dose which is sub therapeutic when given orally or by injection.

In one aspect, the aminosterol 1436 or a salt or derivative thereof is formulated for intranasal administration in a composition which is a dry powder nasal spray or liquid nasal spray.

In one embodiment, the dosage of the aminosterol 1436 or a salt or derivative thereof is escalated every about 3 to about 5 days. In another embodiment, the dose of the aminosterol 1436 or a salt or derivative thereof is escalated about 1×/week, about 2×/week, about every other week, or about 1×/month. In yet another embodiment, the dose of the aminosterol 1436 or a salt or derivative thereof is escalated every about 1, about 2, about 3, about 4, about 5, about 6, about 7, about 8, about 9, about 10, about 11, about 12, about 13, or about 14 days.

In another embodiment, the fixed dose of the aminosterol 1436 or a salt or derivative thereof is given once per day, every other day, once per week, twice per week, three times per week, four times per week, five times per week, six times per week, every other week, or every few days. In addition, the fixed dose of the aminosterol 1436 or a salt or derivative thereof can be administered for a first defined period of time of administration, followed by a cessation of administration for a second defined period of time, followed by resuming administration upon recurrence of SZ or a symptom of SZ. For example, the fixed dose can be incrementally reduced after the fixed dose of aminosterol 1436 or a salt or derivative thereof has been administered to the subject for a period of time. Alternatively, the fixed dose is varied plus or minus a defined amount to enable a modest reduction or increase in the fixed dose. For example, the fixed dose can be increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

In another embodiment, the starting aminosterol 1436 or a salt or derivative thereof dose is higher if the neurodegeneration symptom being evaluated is severe. For example, the starting dose can be based on a baseline score of a cognitive test or tool, wherein if the baseline score correlates with an assessment of mild cognitive impairment, then the starting dose of aminosterol 1436 or a salt or derivative thereof is lower than if the baseline score correlates with an assessment of severe cognitive impairment. In another aspect, a subject experiencing moderate or mild cognitive impairment as determined by a clinical scale or test is administered a starting oral dose of from about 10 to about 75 mg/day; or a subject experiencing severe cognitive impairment as determined by a clinical scale or test is administered a starting oral dose greater than about 75 mg/day.

In one embodiment, the method results in slowing, halting, or reversing progression or onset of neurodegeneration over a defined period of time following administration of the fixed escalated dose of the aminosterol 1436 or a salt or derivative thereof, as measured by a medically-recognized technique. In addition, the method of the invention can result in positively impacting the neurodegeneration, as measured by a medically-recognized technique.

The positive impact and/or progression of neurodegeneration, and/or improvement or resolution of the neurodegeneration symptom being evaluated, may be measured quantitatively or qualitatively by one or more clinically recognized scales, tools, or techniques). Examples of such techniques include computed tomography (CT), magnetic resonance imaging (MRI), magnetic resonance spectroscopy, functional MRI (fMRI), diffusion tensor imaging, single photon emission computed tomography (SPECT), and positron emission tomography (PET). In addition, the progression or onset of neurodegeneration may be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In one embodiment, the fixed escalated dose of the aminosterol 1436 or a salt or derivative thereof reverses dysfunction caused by the neurodegeneration and treats, prevents, improves, and/or resolves the neurodegeneration symptom being evaluated. Again, the improvement or resolution of the neurodegeneration-related symptom can be measured using a clinically recognized scale or tool. Examples of such scales or tools include, for example, the Uniformed Parkinson's Disease Scale (UPDRS), Mini Mental State Examination (MMSE), Mini Mental Parkinson (MMP), Informant Questionnaire on Cognitive Decline in the Elderly (IQCODE), The 7-Minute Screen, Abbreviated Mental Test Score (AMTS), Cambridge Cognitive Examination (CAMCOG), Clock Drawing Test (CDT), General Practitioner Assessment of Cognition (GPCOG), Mini-Cog, Memory Impairment Screen (MIS), Montreal Cognitive Assessment (MoCA), Rowland Universal Dementia Assessment (RUDA), Self-Administered Gerocognitive Examination (SAGE), Short and Sweet Screening Instrument (SAS-SI), Short Blessed Test (SBT), St. Louis Mental Status (SLUMS), Short Portable Mental Status Questionnaire (SPMSQ), Short Test of Mental Status (STMS), Time and Change Test (T&C), Test Your Memory (TYM) test, and Addenbrooke's Cognitive Examination-Revised (ACER). Further, the improvement in the neurodegeneration-related symptom is at least about 3%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale or tool.

In one aspect, the neurodegeneration correlated with abnormal aS pathology and/or dopaminergic dysfunction is related to or correlated with a neurodegenerative disease or neurological disease associated with neural cell death. In another aspect, the neurodegenerative disease or neurological disease or related symptom associated with neural cell death is: (a) selected from the group consisting of septic shock, intracerebral bleeding, subarachnoidal hemorrhage, multiinfarct dementia, inflammatory diseases, neurotrauma, peripheral neuropathies, polyneuropathies, metabolic encephalopathies, and infections of the central nervous system; or (b) selected from the group consisting of synucleopathies, Alzheimer's disease, Parkinson's disease, dementia with Lewy bodies, multiple system atrophy, Huntington's disease, multiple sclerosis, parkinsonism, amyotrophic lateral sclerosis (ALS), schizophrenia, Friedreich's ataxia, vascular dementia, spinal muscular atrophy, frontotemporal dementia, supranuclear palsy, progressive supranuclear palsy, progressive nuclear palsy, degenerative processes associated with aging, dementia of aging, Guadeloupian parkinsonism, spinocerebellar ataxia, hallucinations, stroke, traumatic brain injury, down syndrome, Gaucher's disease, Krabbe's disease (KD), lysosomal conditions affecting glycosphingolipid metabolism, cerebral palsy, and epilepsy.

In another aspect, the neurodegeneration correlated with abnormal αS pathology and/or dopaminergic dysfunction is related to or correlated with a psychological or behavioral disorder. For example, the psychological or behavioral disorder can be selected from the group consisting of aberrant motor and obsessive-compulsive behaviors, sleep disorders, REM sleep behavior disorder (RBD), depression, major depressive disorder, agitation, anxiety, delirium, irritability, ADHD, apathy, bipolar disorder, disinhibition, addiction, illusion and delusions, amnesia, autism,

In one embodiment, the neurodegeneration correlated with abnormal aS pathology and/or dopaminergic dysfunction is related to or correlated with a cerebral ischemic disorder or a general ischemic disorder. For example, the cerebral ischemic disorder can be selected from the group consisting of cerebral microangiopathy, intrapartal cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, and diabetic retinopathy; or the general ischemic disorder can be selected from the group consisting of high blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, and pulmonary edema.

In another embodiment, the neurodegeneration-related symptom is selected from the group consisting of: cognitive impairment (CI) as determined by an IQ score; CI as determined by a memory or cognitive function test; decline in thinking and reasoning skills; confusion; poor motor coordination; loss of short term memory; loss of long term memory; identity confusion; impaired judgement; forgetfulness; depression; anxiety; irritability; obsessive-compulsive behavior; apathy and/or lack of motivation; emotional imbalance; problem solving ability; impaired language; impaired reasoning; impaired decision-making ability; impaired ability to concentrate; impaired communication; impaired ability to conduct routine tasks such as cooking; self-care, including feeding and dressing; constipation; eurodegeneration; sleep problem, sleep disorder, and/or sleep disturbance; hypertension; hypotension; sexual dysfunction; cardiovascular disease; cardiovascular dysfunction; difficulty with working memory; gastrointestinal (GI) disorders; attention deficit and hyperactivity disorder; seizures; urinary dysfunction; difficulty with mastication; vision problems; and muscle weakness.

In one aspect, the neurodegeneration-related symptom to be evaluated is cognitive impairment (CI) as determined by an IQ score or as determined by a memory or cognitive function test and wherein: (a) progression or onset of the CI is slowed, halted, or reversed over a defined period of time following administration of the fixed escalated dose of the aminosterol 1436 or a salt or derivative thereof, as measured by a medically-recognized technique; (b) the CI is positively impacted by the fixed escalated dose of the aminosterol 1436 or a salt or derivative thereof, as measured by a medically-recognized technique; (c) the CI is positively impacted by the fixed escalated dose of the aminosterol 1436 or a salt or derivative thereof, as measured by a medically-recognized technique and the positive impact on and/or progression of cognitive decline is measured quantitatively or qualitatively by one or more medically-recognized techniques selected from the group consisting of ADASCog, Mini-Mental State Exam (MMSE), Mini-cog test, Woodcock-Johnson Tests of Cognitive Abilities, Leiter International Performance Scale, Miller Analogies Test, Raven's Progressive Matrices, Wonderlic Personnel Test, IQ tests, or a computerized tested selected from Cantab Mobile, Cognigram, Cognivue, Cognision, and Automated Neuropsychological Assessment Metrics Cognitive Performance Test (CPT); and/or (d) the progression or onset of CI is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In one embodiment, the neurodegeneration-related symptom to be evaluated is depression and (a) the method results in improvement in a subject's depression, as measured by one or more clinically-recognized depression rating scale; (b) the method results in improvement in a subject's depression, as measured by one or more clinically-recognized depression rating scale and the improvement is in one or more depression characteristics selected from the group consisting of mood, behavior, bodily functions such as eating, sleeping, energy, and sexual activity, and/or episodes of sadness or apathy; and/or (c) the method results in improvement in a subject's depression, as measured by one or more clinically-recognized depression rating scale, and the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%. For example, the one or more clinically-recognized depression rating scale can be selected from the group consisting of the Patient Health Questionnaire-9 (PHQ-9); the Beck Depression Inventory (BDI); Zung Self-Rating Depression Scale; Center for Epidemiologic Studies-Depression Scale (CES-D); and the Hamilton Rating Scale for Depression (HRSD).

In one embodiment, the neurodegeneration-related symptom to be evaluated is constipation, and (a) treating the constipation prevents and/or delays the onset and/or progression of the neurodegeneration; (b) the fixed escalated aminosterol 1436 dose causes the subject to have a bowel movement; (c) the method results in an increase in the frequency of bowel movement in the subject; (d) the method results in an increase in the frequency of bowel movement in the subject and the increase in the frequency of bowel movement is defined as: (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; (e) as a result of the method the subject has the frequency of bowel movement recommended by a medical authority for the age group of the subject; and/or (f) the starting aminosterol 1436 dose is determined by the severity of the constipation, wherein: (i) if the average complete spontaneous bowel movement (CSBM) or spontaneous bowel movement (SBM) is one or less per week, then the starting oral aminosterol 1436 dose is at least about 150 mg; and (ii) if the average CSBM or SBM is greater than one per week, then the starting oral aminosterol 1436 dose is about 75 mg or less.

In one embodiment, the neurodegeneration-related symptom to be evaluated is neurodegeneration correlated with neurodegeneration, and (a) treating the neurodegeneration prevents and/or delays the onset and/or progression of the neurodegeneration; (b) the method results in treating, preventing, and/or delaying the progression and/or onset of neurodegeneration in the subject; (c) progression or onset of the neurodegeneration is slowed, halted, or reversed over a defined period of time following administration of the fixed escalated dose of the aminosterol 1436 or a salt or derivative thereof, as measured by a medically-recognized technique; and/or (d) the neurodegeneration is positively impacted by the fixed escalated dose of the aminosterol 1436 or a salt or derivative thereof, as measured by a medically-recognized technique. For example, the positive impact and/or progression of neurodegeneration can be measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis. In addition, the progression or onset of neurodegeneration can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In one embodiment, the neurodegeneration-related symptom to be evaluated is a sleep problem, sleep disorder, or sleep disturbance and (a) the sleep problem, sleep disorder, or sleep disturbance comprises a delay in sleep onset, sleep fragmentation, REM-behavior disorder, sleep-disordered breathing including snoring and apnea, day-time sleepiness, micro-sleep episodes, narcolepsy, circadian rhythm dysfunction, REM disturbed sleep, or any combination thereof; (b) the sleep problem, sleep disorder, or sleep disturbance comprises REM-behavior disorder, which comprises vivid dreams, nightmares, and acting out the dreams by speaking or screaming, or fidgeting or thrashing of arms or legs during sleep; (c) treating the sleep problem, sleep disorder, or sleep disturbance prevents or delays the onset and/or progression of the neurodegeneration; (d) the method results in a positive change in the sleeping pattern of the subject; wherein the positive change is defined as: (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (f) as a result of the method the subject obtains the total number of hours of sleep recommended by a medical authority for the age group of the subject.

For all of the embodiments described herein, each defined period of time is independently selected from the group consisting of about 1 day to about 10 days, about 10 days to about 30 days, about 30 days to about 3 months, about 3 months to about 6 months, about 6 months to about 12 months, and about greater than 12 months.

### IX. Definitions

The following definitions are provided to facilitate understanding of certain terms used throughout this specification.

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

As used in the description of the invention and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein the term “Aminosterol 1436” encompasses Aminosterol 1436 or a derivative or salt thereof, an isomer or prodrug of Aminosterol 1436.

As used herein, the phrase “therapeutically effective amount” means a dose of Aminosterol 1436, or a salt or derivative thereof, that provides the specific pharmacological effect for which the compound or compounds are being administered. It is emphasized that a therapeutically effective amount will not always be effective in achieving the intended effect in a given subject, even though such dose is deemed to be a therapeutically effective amount by those of skill in the art. For convenience only, exemplary dosages are provided herein. Those skilled in the art can adjust such amounts in accordance with standard practices as needed to treat a specific subject. The therapeutically effective amount may vary based on the route of administration and dosage form, the age and weight of the subject, and/or the severity of the subject's condition. For example one of skill in the art would understand that the therapeutically effective amount for treating a small individual may be different from the therapeutically effective amount for treating a large individual.

The term “administering” as used herein includes prescribing for administration as well as actually administering, and includes physically administering by the subject being treated or by another.

As used herein “subject” or “patient” or “individual” refers to any subject, patient, or individual, and the terms are used interchangeably herein. In this regard, the terms “subject,” “patient,” and “individual” includes mammals, and, in particular humans.

### X. Examples

The following examples are provided to illustrate the present invention. It should be understood, however, that the invention is not to be limited to the specific conditions or details described in these examples. Throughout the specification, any and all references to a publicly available document, including a U.S. patent, are specifically incorporated by reference.

### Example 1

An experiment was conducted to explore the dose response to a delay in growth of B6D2F1 (BDF1) mice to Aminsoterol 1436.

30 day old male BDF1 mice received either vehicle or Aminosterol 1436 intraveneously (IV) at 5 mg/kg or 10 mg/kg (n=15 for each treatment arm) every 3 days until 51 days of age. The Aminosterol 1436 was administered as a aqueous solution in water. BDF1 mice are available from Charles River Laboratories. The animals were fed standard lab chow, offered ad libitum. The animals were weighed and body length measured every 5 days.

As can be seen in FIG. 2, all animals reached the mature weight of about 40 grams. However, while the control animals reached maturity at 120 days, the animals that received 5 mg/kg of Aminosterol 1436 reached maturity at 150 days, corresponding to a 25% delay in reaching maturity, as measured by end weight. In addition, the animals receiving 10 mg/kg reached maturity at 255 days, corresponding to a 112.5% delay in reaching maturity, as measured by end weight. Moreover, as can be seen in FIG. 2, the growth rate of the animals treated at 5 mg/kg was about 30% slower than the controls, while the growth rate of those treated at 10 mg/kg was slowed by 50%, where growth rate is measured by increasing weight on the y axis of FIG. 2. Finally, linear growth was slowed to a corresponding degree (data not shown). Most importantly, both treated and untreated animals reached normal adult dimensions, albeit at different rates.

These results show that administration of Aminosterol 1436 can slow the maturation process of an animal, while the endpoint of maturity, as measured by final weight, remains constant. Moreover, the results also show that the delay in maturation is increased with an increased dose of Aminosterol 1436.

### Example 2

An experiment was conducted to determine whether treatment of animals with Aminosterol 1436 would delay growth of the animal.

C57BL/6 males (12-16 grams) were administered either vehicle or 10 mg/kg (i.p.) of Aminosterol 1436 every 3 days for two doses, for a total of 20 mg/kg over a 6 day period. The mice were about 3 weeks old, and there were 10 mice/arm. The animals were weighed and body length measured once weekly for a period of 40 days.

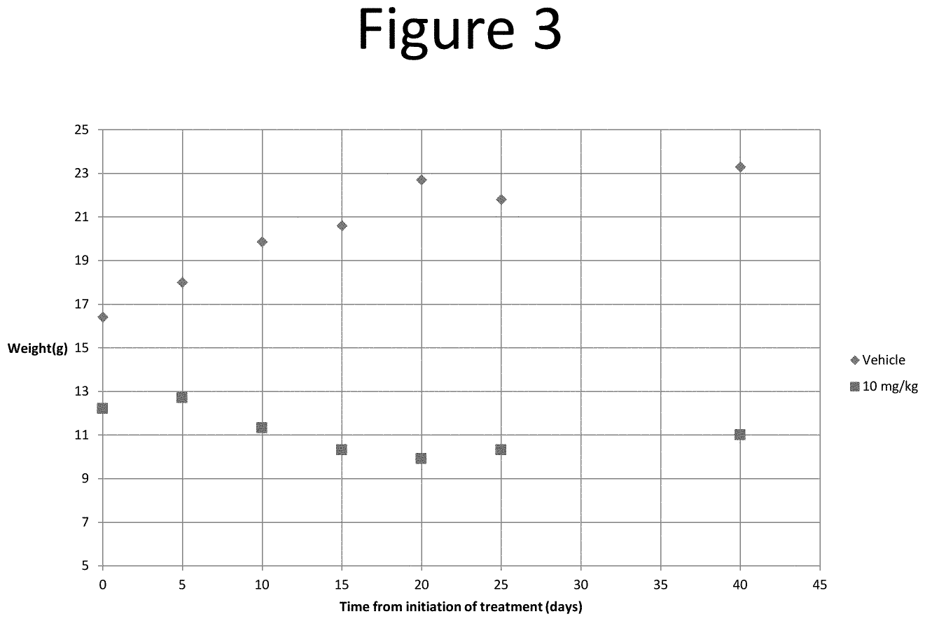

The results shown in FIG. 3 indicate that growth rates of the animals were slowed upon administration of Aminosterol 1436, which is consistent with the results shown in FIG. 2 and described in Example 1. Specifically, at Day 0 animals in the control group had a starting weight (g) of 16 g, while animals in the Aminosterol 1436 group had a weight of 12 g. At day 40, the control group had a weight of 24 g, or an increase of 50%. In contrast, at Day 40 the Aminosterol 1436 group had a weight of 11 g, or a decrease of 8.3%.

These results confirm that administration of Aminosterol 1436 slows the growth rate of animals.

### Example 3

This prophetic example describes an exemplary method of retarding the aging process of a subject. The method comprising administering a pharmaceutical composition comprising a therapeutically effective amount of Aminosterol 1436 or a pharmaceutically acceptable salt or derivative thereof to the subject.

One or more adult human subjects can be given a suitable dosage of Aminosterol 1436 via any pharmaceutically acceptable method, such as oral, intranasal, or injectable. An exemplary daily or weekly dosage can be, for example, about 1 to about 20 mg administered intranasally daily.

The characteristics of aging impacted by administration of Aminosterol 1436 or a derivative or salt thereof that can be measured include muscle endurance, coordination, social behavior and cognitive ability.

First, muscle endurance is measured for each subject prior to initial Aminosterol 1436 dosing to establish a baseline. For example, the partial curl-up test can be used to measure endurance of the abdominal muscles and the push-up test can be used to assess endurance of the upper body. Following initiation of Aminosterol 1436 dosing, the muscle endurance tests are repeated periodically to measure improvement. It is anticipated that muscle endurance will improve following Aminosterol 1436 dosing by about 5% or more.

Coordination can also be evaluated for each subject prior to initial Aminosterol 1436 dosing to establish a baseline by testing the patient's ability to perform rapidly alternating and point-to-point movements correctly. Following initiation of Aminosterol 1436 dosing, the coordination tests are repeated periodically to measure improvement. It is anticipated that coordination will improve following Aminosterol 1436 dosing by about 5% or more.

Cognitive ability can also be evaluated for each subject prior to initial Aminosterol 1436 dosing to establish a baseline using a conventional cognitive ability test. Following initiation of Aminosterol 1436 dosing, the cognitive ability test is repeated periodically to measure improvement. It is anticipated that cognitive ability will improve following Aminosterol 1436 dosing by about 5% or more.

### Example 4

This prophetic example describes an exemplary method of delaying growth and/or maturation of a subject, comprising administering a pharmaceutical composition comprising a therapeutically effective amount of Aminosterol 1436 or a pharmaceutically acceptable salt or derivative thereof to the subject.

One or more juvenile dogs can be given a suitable dosage of Aminosterol 1436 via any pharmaceutically acceptable method, such as oral, intranasal, or injectable. An exemplary daily or weekly dosage can be, for example, about 20 to about 160 mg/m²/day administered daily via any pharmaceutically acceptable route.

The rate of growth of each dog can be measured by recording each dog's height and weight prior to treatment, and then periodically after initiation of treatment. At least one control dog, of the same sex and breed as the tested dogs, does not receive Aminosterol 1436 treatment.

Consistent with the results described in Example 2 and FIG. 2, the treated dogs are expected to show slower growth in terms of height and weight as compared to the untreated dog(s). However, the end point in terms of height and weight of both the treated and untreated dogs is expected to be the same. It is expected that administration of Aminosterol 1436 will result in slowing growth, in terms of height and/or weight, by about 5% or more.

### Example 5

This prophetic example describes an exemplary method of delaying and/or preventing progression and/or onset of age-related neurodegeneration in a subject, comprising administering a pharmaceutical composition comprising a therapeutically effective amount of Aminosterol 1436 or a pharmaceutically acceptable salt or derivative thereof to the subject.

One or more subjects are given a daily dose of a pharmaceutical composition comprising Aminosterol 1436. The composition can be administered via any pharmaceutically acceptable method, such as oral, injectable, or intranasally. In an exemplary method, the composition is administered daily intransally at a dosage of about 1 to about 20 mg.

Neurodegeneration is evaluated prior to treatment to form a baseline, using a medically recognized technique, and then periodically following initiation of treatment. At least one control subject, of the same sex and age as the tested subjects, does not receive Aminosterol 1436 treatment.

The treated subjects are expected to show slowed progression and/or onset of neurodegeneration as compared to the untreated control subject. It is expected that administration of Aminosterol 1436 will result in slowing progression and/or onset of neurodegeneration by about 5% or more.

### Example 6

This prophetic example describes an exemplary method of extending the potential lifespan of a subject, which can be an animal or human.

One or more mice can be given a suitable dosage of Aminosterol 1436 via any pharmaceutically acceptable method, such as oral, intranasal, or injectable. The mice can be juveniles or adults. An exemplary daily or weekly dosage can be, for example, about 1 to about 10 mg/kg every 3 days administered via IPeritoneal or INasal. A control group of mice, of the same sex, are not treated.

The lifespan of each mouse is measured and compared to that of the control group. It is expected that administration of Aminosterol 1436 will result in extending the lifespan of the mice by about 5% or more as compared to the control.

It will be apparent to those skilled in the art that various modifications and variations can be made in the methods and compositions of the present invention without departing from the spirit or scope of the invention. Thus, it is intended that the present invention cover the modifications and variations of this invention, provided they come within the scope of the appended claims and their equivalents.

### REFERENCES

Ahima et al., “Appetite suppression and weight reduction by a centrally active aminosterol,” Diabetes, 51: 2099-2104 (2002).
Blagosklonny, M. V., “Big mice die young but large animals live longer,” Aging (Albany N.Y.) 5, 227-233 (2013).
Lantz et al., “Inhibition of PTP1B by trodusquemine (MSI-1436) causes fat-specific weight loss in diet-induced obese mice,” Obesity (Silver Spring), 18:1516-1523 (2010).
Miller et al., “Big mice die young: early life body weight predicts longevity in genetically heterogeneous mice,” Aging Cell, 1: 22-29 (2002).
Vanhooren, V., Libert, C., “The mouse as a model organism in aging research: usefulness, pitfalls and possibilities,” Ageing Res. Rev., 12: 8-21 (2013).
T. Hicklin, “Brain cells that influence aging,” NIH Research Matters (Aug. 15, 2017), https://www.nih.gov/news-events/nih-research-matters/brain-cells-influence-aging (accessed on Feb. 14, 2018).
Zhang et al., “Hypothalamic stem cells control ageing speed partly through exosomal miRNAs,” Nature, 548(7665):52-57 (2017).
Zhang et al., “Hypothalamic programming of systemic ageing involving IKK-β, NF-κB and GnRH,” Nature, 497:211-216 (2013).

We claim:

1. A method of treating, preventing, and/or delaying the progression and/or onset of neurodegeneration in a subject in need, comprising administering to the subject a pharmaceutical composition comprising a therapeutically effective amount of Aminosterol 1436 or a pharmaceutically acceptable salt or derivative thereof.

2. The method of claim 1, wherein the neurodegeneration:
(a) is age-related; and/or
(b) is correlated with one or more conditions or diseases selected from the group consisting of age-related dementia, Alzheimer's disease, Parkinson's disease, Lewy Body dementia, fronto temperal dementia, vascular dementia, amyotrophic lateral sclerosis (ALS), multiple sclerosis (MS), multiple system atrophy (MSA), progressive supranuclear palsy (PSP)), olivo-ponto-cerebellar degeneration, and age related cognitive decline without a specific diagnosis from the group above.

3. The method of claim 1, wherein:
(a) progression or onset of the neurodegeneration is slowed, halted, or reversed over a defined time period following administration of the pharmaceutical composition, as measured by a medically-recognized technique; and/or
(b) the neurodegeneration is positively impacted by administration of the pharmaceutical composition; and/or
(c) the neurodegeneration is positively impacted by administration of the pharmaceutical composition and the positive impact and/or progression of neurodegeneration is measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; and/or
(d) progression or onset of the neurodegeneration is slowed, halted, or reversed over a defined time period following administration of the pharmaceutical composition, as measured by a medically-recognized technique, and the progression or onset of neurodegeneration is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(e) the neurodegeneration is correlated with abnormal α-synuclein (αS) pathology and/or dopaminergic dysfunction.

4. The method of claim 1, wherein the neurodegeneration is correlated with:
(a) neural cell death caused by septic shock, intracerebral bleeding, subarachnoidal hemorrhage, multiinfarct dementia, inflammatory diseases, neurotrauma, peripheral neuropathies, polyneuropathies, metabolic encephalopathies, and infections of the central nervous system; or
(b) a neurodegenerative disease selected from the group consisting of synucleopathies, Alzheimer's disease, Parkinson's disease, dementia with Lewy bodies, multiple system atrophy, Huntington's disease, multiple sclerosis, parkinsonism, amyotrophic lateral sclerosis (ALS), schizophrenia, Friedreich's ataxia, vascular dementia, spinal muscular atrophy, frontotemporal dementia, supranuclear palsy, progressive supranuclear palsy, progressive nuclear palsy, degenerative processes associated with aging, dementia of aging, Guadeloupian parkinsonism, spinocerebellar ataxia, hallucinations, stroke, traumatic brain injury, down syndrome, Gaucher's disease, Krabbe's disease (KD), lysosomal conditions affecting glycosphingolipid metabolism, cerebral palsy, and epilepsy; or
(c) a psychological or behavioral disorder; or
(d) a psychological or behavioral disorder which is selected from the group consisting of aberrant motor and obsessive-compulsive behaviors, sleep disorders, REM sleep behavior disorder (RBD), depression, major depressive disorder, agitation, anxiety, delirium, irritability, ADHD, apathy, bipolar disorder, disinhibition, addiction, illusion and delusions, amnesia, and autism; or
(e) a cerebral ischemic disorder or a general ischemic disorder; or
(f) a cerebral ischemic disorder which is selected from the group consisting of cerebral microangiopathy, intrapartal cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, and diabetic retinopathy; or
(g) a general ischemic disorder which is selected from the group consisting of high blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, and pulmonary edema.

5. A method of reversibly slowing or delaying the growth, maturation, and/or aging of a subject, and/or extending the potential lifespan of the subject, comprising administering to the subject a pharmaceutical composition comprising a therapeutically effective amount of Aminosterol 1436 or a pharmaceutically acceptable salt or derivative thereof.

6. The method of claim 5, wherein:
(a) the slowed or delayed growth is measured by height and/or weight, as compared to a subject the same age and sex, who is not administered the pharmaceutical composition comprising a therapeutically effective amount of Aminosterol 1436 or a pharmaceutically acceptable salt or derivative thereof; and/or
(b) the subject administered a pharmaceutical composition according to the invention has delayed or slowed growth, as measured by height and/or weight, as compared to a subject the same age and sex and who is not treated with a method of the invention, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(c) the delayed maturation is measured by showing a delay in skeletal maturation; and/or
(d) the subject administered the pharmaceutical composition has delayed maturation, as measured by skeletal maturation, as compared to an untreated subject which is the same age and sex, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

7. The method of claim 5, where the treatment is administered during a critical “developmental window” of the subject, which is optionally prior to the onset of maturity of the subject.

8. The method claim 5, wherein:
(a) the characteristics of aging impacted by administration of the pharmaceutical composition are selected from the group consisting of muscle endurance, coordination, social behavior and cognitive ability; and/or
(b) administration of the pharmaceutical composition improves impaired muscle endurance, as compared to an untreated subject which is the same sex and age, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(c) administration of the pharmaceutical composition improves impaired coordination, as compared to an untreated subject which is the same sex and age, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(d) administration of the pharmaceutical composition improves impaired cognitive ability, as compared to an untreated subject which is the same sex and age, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

9. A method of treating, preventing, or delaying the onset of age-related diseases, conditions or health problems in a subject, comprising administering a pharmaceutical composition comprising a therapeutically effective amount of Aminosterol 1436 or a pharmaceutically acceptable salt or derivative thereof to the subject.

10. The method of claim 9, wherein the age-related disease, condition, or health problem is selected from the group consisting of atherosclerosis and cardiovascular disease, cancer, arthritis, cataracts, osteoporosis, diabetes, hypertension, Alzheimer's disease, arthritis, and osteoporosis.

11. The method of claim 1, wherein:
(a) the aminosterol 1436 or a salt or derivative thereof is a pharmaceutically acceptable grade of the aminosterol 1436 or a salt or derivative thereof; and/or
(b) the pharmaceutical composition is administered via any pharmaceutically acceptable method; and/or
(c) the pharmaceutical composition is administered intravenously, intradermally, subcutaneously, orally, rectally, sublingually, intrathecally, intranasally, or by inhalation; and/or
(d) the pharmaceutical composition is administered intranasally; and/or
(e) the pharmaceutical composition is formulated for oral administration in a composition which is a liquid, capsule, or tablet designed to disintegrate in either the stomach, upper small intestine, or more distal portions of the intestine; and/or
(f) the pharmaceutical composition is formulated for intranasal administration in a composition which is a dry powder nasal spray or liquid nasal spray; and/or
(g) the pharmaceutical composition is formulated into a dosage form selected from the group consisting of liquid dispersions, gels, aerosols, lyophilized formulations, tablets, and capsules; and/or
(h) the pharmaceutical composition is formulated into a dosage form selected from the group consisting of controlled release formulations, fast melt formulations, delayed release formulations, extended release formulations, pulsatile release formulations, and mixed immediate release and controlled release formulations.

12. The method of claim 1, wherein:
(a) the dosage of Aminosterol 1436 or a derivative or salt thereof is selected from the group consisting of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 20, 25, 30, 35, 40, 45, and 50 mg/kg; and/or
(b) the dosage of Aminosterol 1436 or a derivative or salt thereof is selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, or 110 mg/m²; and/or
(c) the dosage of Aminosterol 1436 or a derivative or salt thereof is selected from the group consisting of about 10 mg to about 400 mg, or about 50 mg to about 350 mg, or about 100 mg to about 300 mg, or about 100 mg to about 200 mg.

13. The method of claim 1, further comprising:
(a) determining a dose of the aminosterol 1436 or a salt or derivative thereof for the subject, wherein the aminosterol 1436 dose is determined based on the effectiveness of the aminosterol 1436 dose in improving or resolving a symptom being evaluated, wherein the symptom is related to neurodegeneration, age-related diseases, and/or growth, maturation, and/or aging of the subject; and
(b) followed by administering the dose of the aminosterol 1436 or a salt or derivative thereof to the subject for a defined period of time, wherein the method comprises:
(i) identifying a symptom to be evaluated;
(ii) identifying a starting dose of the aminosterol 1436 or a salt or derivative thereof for the subject; and
(iii) administering an escalating dose of the aminosterol 1436 or a salt or derivative thereof to the subject over a defined period of time until an effective dose is identified, wherein the effective dose is the dose where improvement of the symptom is observed, and fixing the aminosterol 1436 dose at that level in that particular subject.

14. The method of claim 13, wherein:
(a) the dose of the aminosterol 1436 or a salt or derivative thereof reverses dysfunction caused by the neurodegeneration and treats, prevents, improves, and/or resolves the symptom being evaluated; and/or
(b) the improvement or resolution of the symptom is measured using a clinically recognized scale or tool; and/or
(c) the improvement or resolution of the symptom is measured using a clinically recognized scale or tool and the clinical scale or tool is selected from the group consisting of Uniformed Parkinson's Disease Scale (UPDRS), Mini Mental State Examination (MMSE), Mini Mental Parkinson (MMP), Informant Questionnaire on Cognitive Decline in the Elderly (IQCODE), The 7-Minute Screen, Abbreviated Mental Test Score (AMTS), Cambridge Cognitive Examination (CAMCOG), Clock Drawing Test (CDT), General Practitioner Assessment of Cognition (GPCOG), Mini-Cog, Memory Impairment Screen (MIS), Montreal Cognitive Assessment (MoCA), Rowland Universal Dementia Assessment (RUDA), Self-Administered Gerocognitive Examination (SAGE), Short and Sweet Screening Instrument (SAS-SI), Short Blessed Test (SBT), St. Louis Mental Status (SLUMS), Short Portable Mental Status Questionnaire (SPMSQ), Short Test of Mental Status (STMS), Time and Change Test (T&C), Test Your Memory (TYM) test, and Addenbrooke's Cognitive Examination-Revised (ACER); and/or
(d) the improvement in the symptom is at least about 3%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale or tool.

15. The method of claim 13, wherein the aminosterol 1436 or a salt or derivative thereof is administered orally and:
(a) the starting dose ranges from about 1 mg up to about 175 mg/day;
(b) the starting oral dose is about 25 mg/day;
(c) the dose of the for the subject following dose escalation is fixed at a range of from about 1 mg up to about 500 mg/day;
(d) the dose of the following dose escalation is fixed at a dose of about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 mg/day;
(e) the starting oral aminosterol 1436 dose is about 10, about 15, about 20, about 25, about 30, about 35, about 40, about 45, about 60, about 65, about 70, or about 75 mg/day; and/or
(f) the dose of the aminosterol 1436 or a salt or derivative thereof is escalated in about 25 mg increments.

16. The method claim 13, wherein the aminosterol 1436 or a salt or derivative thereof is administered intranasally and:
(a) the starting dose ranges from about 0.001 mg to about 3 mg/day;
(b) the dose for the subject following escalation is fixed at a range of from about 0.001 mg up to about 6 mg/day;
(c) the dose following escalation is a dose which is subtherapeutic when administered orally or by injection; and/or
(d) the dose is escalated in increments of about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

17. The method of claim 13, wherein the dose of the aminosterol 1436 or a salt or derivative thereof:
(a) is escalated every about 3 to about 5 days; and/or
(b) is escalated every about 1 to about 14 days; and/or
(c) is escalated every about 1, about 2, about 3, about 4, about 5, about 6, about 7, about 8, about 9, about 10, about 11, about 12, about 13, or about 14 days; and/or
(d) is escalated about 1×/week, about 2×/week, about every other week, or about 1×/month; and/or
(e) the dose, including the starting or fixed dose, of the aminosterol 1436 or a salt or derivative thereof is administered once per day, every other day, once per week, twice per week, three times per week, four times per week, five times per week, six times per week, every other week, or every few days; and/or
(f) the dose, including the starting or fixed dose, of the aminosterol 1436 or a salt or derivative thereof is administered for a first defined period of time of administration, followed by a cessation of administration for a second defined period of time, followed by resuming administration upon recurrence of neurodegeneration or a symptom of neurodegeneration; and/or
(g) the dose, including the starting or fixed dose, of the aminosterol 1436 or a salt or derivative thereof is incrementally reduced after the fixed dose of aminosterol or a salt or derivative thereof has been administered to the subject for a defined period of time; and/or
(h) the dose, including the starting or fixed dose, of the aminosterol 1436 or a salt or derivative thereof is varied plus or minus a defined amount to enable a modest reduction or increase in the fixed dose; and/or
(i) the dose, including the starting or fixed dose, of the aminosterol 1436 or a salt or derivative thereof is varied plus or minus a defined amount to enable a modest reduction or increase in the fixed dose, and the fixed aminosterol 1436 dose is increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%; and/or
(j) the starting dose of the aminosterol 1436 or a salt or derivative thereof is higher if the symptom being evaluated is severe, as measured using a clinically recognized scale or tool; and/or
(k) the starting aminosterol 1436 dose is based on a baseline score of a cognitive test or tool, wherein if the baseline score correlates with an assessment of mild neurodegeneration, then the starting aminosterol 1436 dose is lower than if the baseline score correlates with an assessment of severe neurodegeneration; and/or
(l) the subject experiences moderate or mild neurodegeneration as determined by a clinical scale or test, and wherein the starting oral aminosterol 1436 dose is from about 10 to about 75 mg/day; and/or
(m) the subject experiences severe neurodegeneration as determined by a clinical scale or test, and wherein the starting oral aminosterol 1436 dose is greater than about 75 mg/day.

18. The method of claim 13, wherein the symptom is selected from the group consisting of:
(a) cognitive impairment as determined by an IQ score;
(b) cognitive impairment as determined by a memory or cognitive function test;
(c) decline in thinking and reasoning skills;
(d) confusion;
(e) poor motor coordination;
(f) loss of short term memory;
(g) loss of long term memory;
(h) identity confusion;
(i) impaired judgement;
(j) forgetfulness;
(k) depression;
(l) anxiety;
(m) irritability;
(n) obsessive-compulsive behavior;
(o) apathy and/or lack of motivation;
(p) emotional imbalance;
(q) problem solving ability;
(r) impaired language;
(s) impaired reasoning;
(t) impaired decision-making ability;
(u) impaired ability to concentrate;
(v) impaired communication;
(w) impaired ability to conduct routine tasks such as cooking;
(x) self-care, including feeding and dressing;
(y) constipation;
(z) neurodegeneration;
(aa) sleep problem, sleep disorder, and/or sleep disturbance;
(bb) hypertension;
(cc) hypotension;
(dd) sexual dysfunction;
(ee) cardiovascular disease;
(ff) cardiovascular dysfunction;
(gg) difficulty with working memory;
(hh) gastrointestinal (GI) disorders;
(ii) attention deficit and hyperactivity disorder;
(jj) seizures;
(kk) urinary dysfunction;
(ll) difficulty with mastication;
(mm) vision problems; and
(nn) muscle weakness.

19. The method of claim 18, wherein the symptom to be evaluated is:
(a) cognitive impairment as determined by an IQ score or as determined by a memory or cognitive function test and wherein:
(i) progression or onset of the CI is slowed, halted, or reversed over a defined period of time following administration of the fixed escalated dose of the aminosterol 1436 or a salt or derivative thereof, as measured by a medically-recognized technique;
(ii) the CI is positively impacted by the fixed escalated dose of the aminosterol 1436 or a salt or derivative thereof, as measured by a medically-recognized technique;
(iii) the CI is positively impacted by the fixed escalated dose of the aminosterol 1436 or a salt or derivative thereof, as measured by a medically-recognized technique and the positive impact on and/or progression of cognitive decline is measured quantitatively or qualitatively by one or more medically-recognized techniques selected from the group consisting of ADASCog, Mini-Mental State Exam (MMSE), Mini-cog test, Woodcock-Johnson Tests of Cognitive Abilities, Leiter International Performance Scale, Miller Analogies Test, Raven's Progressive Matrices, Wonderlic Personnel Test, IQ tests, or a computerized tested selected from Cantab Mobile, Cognigram, Cognivue, Cognision, and Automated Neuropsychological Assessment Metrics Cognitive Performance Test (CPT); and/or
(iv) the progression or onset of CI is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; or
(b) depression, wherein:
(i) the method results in improvement in a subject's depression, as measured by one or more clinically-recognized depression rating scale;
(ii) the method results in improvement in a subject's depression, as measured by one or more clinically-recognized depression rating scale and the improvement is in one or more depression characteristics selected from the group consisting of mood, behavior, bodily functions such as eating, sleeping, energy, and sexual activity, and/or episodes of sadness or apathy; and/or
(iii) the method results in improvement in a subject's depression, as measured by one or more clinically-recognized depression rating scale, and the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%,
and optionally wherein the one or more clinically-recognized depression rating scale is selected from the group consisting of the Patient Health Questionnaire-9 (PHQ-9); the Beck Depression Inventory (BDI); Zung Self-Rating Depression Scale; Center for Epidemiologic Studies-Depression Scale (CES-D); and the Hamilton Rating Scale for Depression (HRSD);
(c) constipation, wherein:
(i) treating the constipation prevents and/or delays the onset and/or progression of the neurodegeneration;
(ii) the fixed escalated aminosterol 1436 dose causes the subject to have a bowel movement;
(iii) the method results in an increase in the frequency of bowel movement in the subject;
(iii) the method results in an increase in the frequency of bowel movement in the subject and the increase in the frequency of bowel movement is defined as:
(1) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(2) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%;
(iv) as a result of the method the subject has the frequency of bowel movement recommended by a medical authority for the age group of the subject; and/or
(v) the starting aminosterol 1436 dose is determined by the severity of the constipation, wherein:
(1) if the average complete spontaneous bowel movement (CSBM) or spontaneous bowel movement (SBM) is one or less per week, then the starting aminosterol 1436 dose is at least about 150 mg; and
(2) if the average CSBM or SBM is greater than one per week, then the starting aminosterol 1436 dose is about 75 mg or less;
(d) a sleep problem, sleep disorder, or sleep disturbance and:
(i) the sleep problem, sleep disorder, or sleep disturbance comprises a delay in sleep onset, sleep fragmentation, REM-behavior disorder, sleep-disordered breathing including snoring and apnea, day-time sleepiness, micro-sleep episodes, narcolepsy, circadian rhythm dysfunction, REM disturbed sleep, or any combination thereof;
(ii) the sleep problem, sleep disorder, or sleep disturbance comprises REM-behavior disorder, which comprises vivid dreams, nightmares, and acting out the dreams by speaking or screaming, or fidgeting or thrashing of arms or legs during sleep;
(iii) treating the sleep problem, sleep disorder, or sleep disturbance prevents or delays the onset and/or progression of the CI;
(iv) the method results in a positive change in the sleeping pattern of the subject; wherein the positive change is defined as:
(1) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(2) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(v) as a result of the method the subject obtains the total number of hours of sleep recommended by a medical authority for the age group of the subject.
(e) the method of any one of subsections (a)-(d), wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

20. The method of claim 1, wherein the subject is a human.
